SEQUENCE LISTING (8 sequences)

SEQ ID NO: 1 - DNA, 72 bp, Artificial Sequence
  Description of Artificial Sequence Synthetic oligonucleotide
  modified_base (30)..(39): a, c, t, g, unknown or other
aatgatacggcgaccaccgagatctacacnnnnnnnnnnacactctttccctacacgacgctcttccgatct

SEQ ID NO: 2 - DNA, 65 bp, Artificial Sequence
  Description of Artificial Sequence Synthetic oligonucleotide
gatcggaagagcacacgtctgaactccagtcactggagtagatctcgtatgccgtcttctgcttg

SEQ ID NO: 3 - DNA, 28 bp, Artificial Sequence
  Description of Artificial Sequence Synthetic oligonucleotide
aatgatacggcgaccaccgagatctaca

SEQ ID NO: 4 - DNA, 24 bp, Artificial Sequence
  Description of Artificial Sequence Synthetic oligonucleotide
caagcagaagacggcatacgagat

SEQ ID NO: 5 - DNA, 82 bp, Artificial Sequence
  Description of Artificial Sequence Synthetic oligonucleotide
aatgatacggcgaccaccgagatctacacbbbbbabbabbabababaabacactctttccctacacgacgctcttccgatct

SEQ ID NO: 6 - DNA, 77 bp, Artificial Sequence
  Description of Artificial Sequence Synthetic oligonucleotide
gatcggaagagcacacgtctgaactccagtcacbbbbbabbabbabababaabatctcgtatgccgtcttctgcttg

SEQ ID NO: 7 - DNA, 168 bp, Artificial Sequence
  Description of Artificial Sequence Synthetic polynucleotide
  modified_base (83)..(90): a, c, t, g, unknown or other
aatgatacggcgaccaccgagatctacacaaaaaaaaaaaaaaaaaaaaacactctttccctacacgacgctcttccgatctnnnnnnnnagatcggaagagcacacgtctgaactccagtcacaaaaaaaaaaaaaaaaaaaaatctcgtatgccgtcttctgcttg

SEQ ID NO: 8 - DNA, 169 bp, Artificial Sequence
  Description of Artificial Sequence Synthetic polynucleotide
  modified_base (79)..(87): a, c, t, g, unknown or other
caagcagaagacggcatacgagatttttttttttttttttttttgtgactggagttcagacgtgtgctcttccgatctnnnnnnnnnagatcggaagagcgtcgtgtagggaaagagtgtttttttttttttttttttttgtgtagatctcggtggtcgccgtatcatt

(19) United States
(12) Patent Application Publication
(10) Pub. No.: US 2023/0151356 A1
(43) Pub. Date: May 18, 2023

(54) Floating Barcodes

(71) Applicant: Personal Genome Diagnostics Inc., Baltimore, MD (US)

(72) Inventor: John F. Thompson, Warwick, RI (US)

(21) Appl. No.: 17/916,938

(22) PCT Filed: Apr. 6, 2021
(86) PCT No.: PCT/US2021/026043

§ 371 Date: Oct. 4, 2022

Related U.S. Application Data
(60) Provisional application No. 63/006,556, filed on Apr. 7, 2020.

(51) Int. Cl. C12N 15/10; C12Q 1/6806

(52) U.S. Cl. CPC C12N 15/1065; C12Q 1/6806

(57) ABSTRACT

Provided herein are systems and sets of oligonucleotides for labeling and analyzing nucleic acid molecules that include index barcodes with pre-determined numbers of index positions. Also provided herein are methods for labeling and analyzing nucleic acid molecules, as well as methods of identifying erroneous sequence reads using the sample and molecular barcodes described herein.

### CROSS-REFERENCE TO RELATED APPLICATIONS

This application claims the benefit of priority under 35 U.S.C. § 119(e) OF U.S. Provisional Application No. 63/006,556, filed Apr. 7, 2020. The disclosure of the prior application is considered part of and is incorporated by reference in the disclosure of this application.

### INCORPORATION OF SEQUENCE LISTING

The material in the accompanying sequence listing is hereby incorporated by reference into this application. The accompanying sequence listing text file, named PGDX3120-1WO_SL.txt, was created on Mar. 31, 2021, and is 11 kb. The file can be accessed using Microsoft Word on a computer that uses Windows OS.

### BACKGROUND OF THE INVENTION

### Field of the Invention

The invention relates generally to nucleic acid sequences and more specifically to sequences, referred to as barcodes, for labeling and analyzing nucleic acid molecules.

### Background Information

Barcodes are often used to tag nucleic acids such as DNA or RNA molecules being sequenced to identify their source. Barcodes can be used to mark a sample, cell, or other origin of the DNA or RNA molecule. A barcode can provide information about where the molecule came from and whether a particular molecule may have been sequenced multiple times in a pool due to amplification. Often, multiple pieces of information are desired, such as the sample and molecular origin. The more complex the source, the more challenging it is to create a sufficient number of barcodes and/or reads of barcodes with certainty of having the correct sequence and avoiding misassignment of source. Specifically, an insufficient number of barcodes and difficulties in correcting sequence errors in complex barcodes limit genomic analysis of nucleic acid molecules, such as nucleic acids from pooled samples, for example. Thus, there exists a need for novel systems and methods of barcoding nucleic acids that allow for multiplex genomic analysis of nucleic acids and improved error correction to minimize incorrect assignment and loss of sequence reads resulting from barcode sequence uncertainty.

### SUMMARY OF THE INVENTION

The present invention relates to systems and sets of oligonucleotides for labeling and analyzing nucleic acid molecules that include index “barcodes” with pre-determined numbers of index positions. Methods for labeling and analyzing nucleic acid molecules are also provided.

In one embodiment, the invention provides systems for labeling nucleic acid molecules in a sample including: a set of oligonucleotides including a plurality of barcodes, each barcode including a stretch of contiguous bases including: (i) a sample barcode including a pre-determined number of sample index positions including one or more specific nucleotides, wherein the location of sample index positions varies between samples; and (ii) a molecular barcode including molecular index positions including a nucleotide that differs from the nucleotides at sample index positions, wherein sample index positions are interspersed among molecular index positions. In one aspect, the pre-determined number of sample barcode positions can vary among different sample barcodes in systems for labeling nucleic acids provided herein. In some aspects, the barcode includes about 10 to about 35 nucleotides. In other aspects, the barcode includes about 12 to about 25 nucleotides. In another aspect, the sample barcode includes 2, 3, 4, 5, 6, 7, 8, 9, 10, 11, 12, 13, 14, 15, 16, 17, 18, 19, or 20 sample index positions, or a combination thereof. In some aspects, the sample barcode includes about 4 to about 12 sample index positions. In other aspects, the molecular barcode includes about 5 to about 25 molecular index positions. In various aspects, the molecular barcode includes about 5 to about 15 molecular index positions. In one aspect, sample index position nucleotides and molecular index position nucleotides are selected from: (A) the sample index position nucleotide is A and the molecular index position nucleotide is C, G, T, or a combination thereof; (B) the sample index position nucleotide is T and the molecular index position nucleotide is C, G, A, or a combination thereof; (C) the sample index position nucleotide is C and the molecular index position nucleotide is G, A, T, or a combination thereof; (D) the sample index position nucleotide is G and the molecular index position nucleotide is C, A, T, or a combination thereof; (E) the sample index position nucleotide is A, T, or a combination thereof and the molecular index position nucleotide is C, G, or a combination thereof (F) the sample index position nucleotide is A, C, or a combination thereof and the molecular index position nucleotide is T, G, or a combination thereof (G) the sample index position nucleotide is A, G, or a combination thereof and the molecular index position nucleotide is T, C, or a combination thereof (H) the sample index position nucleotide is T, C, or a combination thereof and the molecular index position nucleotide is A, G, or a combination thereof; (I) the sample index position nucleotide is T, G, or a combination thereof and the molecular index position nucleotide is A, C, or a combination thereof; or (J) the sample index position nucleotide is G, C, or a combination thereof and the molecular index position nucleotide is A, T, or a combination thereof. In some aspects, each barcode includes one or more additional index barcodes including index positions. In many aspects, the one or more additional index barcode is a cellular barcode, a barcode that provides a measure of DNA length of an unrepaired end, or both a cellular barcode and a barcode that provides a measure of DNA length of an unrepaired end. In other aspects, each oligonucleotide in the set of oligonucleotides further includes non-barcode positions including sites for hybridization, sites for sequence primer binding, sites for amplification, or any combination thereof.

In another embodiment, the invention provides sets of oligonucleotides for labeling nucleic acid molecules in a sample including a plurality of barcodes, each barcode including: (i) a sample barcode including a pre-determined number of sample index positions including one or more specific nucleotides, wherein the location of sample index positions varies between samples; and (ii) a molecular barcode including molecular index positions including a nucleotide that differs from the nucleotides at sample index positions, wherein sample index positions and molecular index positions are interspersed in a stretch of contiguous bases. In one aspect, the pre-determined number of sample barcode positions varies among different sample barcodes. In some aspects, the barcode includes about 10 to about 35 nucleotides. In other aspects, the barcode includes about 12 to about 25 nucleotides. In another aspect, the sample barcode includes 2, 3, 4, 5, 6, 7, 8, 9, 10, 11, 12, 13, 14, 15, 16, 17, 18, 19, or 20 sample index positions, or a combination thereof. In some aspects, the sample barcode includes about 4 to about 12 sample index positions. In one aspect, the molecular barcode includes about 5 to about 25 molecular index positions. In some aspects, the molecular barcode includes about 5 to about 15 molecular index positions. In other aspects, sample index position nucleotides and molecular index position nucleotides are selected from: (A) the sample index position nucleotide is A and the molecular index position nucleotide is C, G, T, or a combination thereof; (B) the sample index position nucleotide is T and the molecular index position nucleotide is C, G, A, or a combination thereof; (C) the sample index position nucleotide is C and the molecular index position nucleotide is G, A, T, or a combination thereof; (D) the sample index position nucleotide is G and the molecular index position nucleotide is C, A, T, or a combination thereof; (E) the sample index position nucleotide is A, T, or a combination thereof and the molecular index position nucleotide is C, G, or a combination thereof (F) the sample index position nucleotide is A, C, or a combination thereof and the molecular index position nucleotide is T, G, or a combination thereof (G) the sample index position nucleotide is A, G, or a combination thereof and the molecular index position nucleotide is T, C, or a combination thereof (H) the sample index position nucleotide is T, C, or a combination thereof and the molecular index position nucleotide is A, G, or a combination thereof; (I) the sample index position nucleotide is T, G, or a combination thereof and the molecular index position nucleotide is A, C, or a combination thereof; or (J) the sample index position nucleotide is G, C, or a combination thereof and the molecular index position nucleotide is A, T, or a combination thereof. In some aspects, each barcode includes one or more additional index barcodes including index positions. In many aspects, the one or more additional index barcode is a cellular barcode, a barcode that provides a measure of DNA length of an unrepaired end, or both a cellular barcode and a barcode that provides a measure of DNA length of an unrepaired end. In some aspects, each oligonucleotide in a set of oligonucleotides further includes non-barcode positions including sites for hybridization, sites for sequence primer binding, sites for amplification, or any combination thereof.

In an additional embodiment, the invention provides methods for analyzing sequences of nucleic acid molecules in a sample including: (a) attaching a plurality of oligonucleotides to the nucleic acid molecules, wherein each oligonucleotide includes a barcode including: (i) a sample barcode including a pre-determined number of sample index positions including one or more specific nucleotides, wherein the location of sample index positions varies between samples; and (ii) a molecular barcode including molecular index positions including a nucleotide that differs from the nucleotides at sample index positions, wherein sample index positions and molecular index positions are interspersed in a stretch of contiguous bases; and (b) sequencing the nucleic acid molecules, wherein sequence reads include barcode sequences. In one aspect, the methods for analyzing sequences of nucleic acid molecules in a sample provided herein can further include attaching an oligonucleotide including the same sample barcode to each end of a nucleic acid molecule in the sample. In another aspect, the pre-determined number of sample barcode positions varies among different sample barcodes. In some aspects, the barcode includes about 10 to about 35 nucleotides. In other aspects, the barcode includes about 12 to about 25 nucleotides. In some aspects, the sample barcode includes 2, 3, 4, 5, 6, 7, 8, 9, 10, 11, 12, 13, 14, 15, 16, 17, 18, 19, or 20 sample index positions, or a combination thereof. In other aspects, the sample barcode includes about 4 to about 12 sample index positions. In one aspect, the molecular barcode includes about 5 to about 25 molecular index positions. In some aspects, the molecular barcode includes about 5 to about 15 molecular index positions. In one aspect, sample index position nucleotides and molecular index position nucleotides are selected from: (A) the sample index position nucleotide is A and the molecular index position nucleotide is C, G, T, or a combination thereof; (B) the sample index position nucleotide is T and the molecular index position nucleotide is C, G, A, or a combination thereof; (C) the sample index position nucleotide is C and the molecular index position nucleotide is G, A, T, or a combination thereof; (D) the sample index position nucleotide is G and the molecular index position nucleotide is C, A, T, or a combination thereof; (E) the sample index position nucleotide is A, T, or a combination thereof and the molecular index position nucleotide is C, G, or a combination thereof (F) the sample index position nucleotide is A, C, or a combination thereof and the molecular index position nucleotide is T, G, or a combination thereof (G) the sample index position nucleotide is A, G, or a combination thereof and the molecular index position nucleotide is T, C, or a combination thereof (H) the sample index position nucleotide is T, C, or a combination thereof and the molecular index position nucleotide is A, G, or a combination thereof; (I) the sample index position nucleotide is T, G, or a combination thereof and the molecular index position nucleotide is A, C, or a combination thereof; or (J) the sample index position nucleotide is G, C, or a combination thereof and the molecular index position nucleotide is A, T, or a combination thereof. In other aspects, each barcode includes one or more additional index barcodes including index positions. In some aspects, the one or more additional index barcode is a cellular barcode, a barcode that provides a measure of DNA length of an unrepaired end, or both a cellular barcode and a barcode that provides a measure of DNA length of an unrepaired end. In some aspects, methods for analyzing sequences of nucleic acid molecules in a sample provided herein further include assigning the sequence reads to sample families based on the location of sample index positions. In other aspects, methods for analyzing sequences of nucleic acid molecules in a sample provided herein further include assigning the sequence reads to molecular families based on the location of molecular index positions and the nucleotide at each molecular index position. In some aspects, methods for analyzing sequences of nucleic acid molecules in a sample provided herein further include correcting for sequencing errors by comparing the number and location of sample index positions in a sequence read to the pre-determined number and location of sample index positions. In other aspects, methods for analyzing sequences of nucleic acid molecules in a sample provided herein further include correcting for sequencing errors by comparing sample barcodes at both ends of a sequence read. In some aspects, methods for analyzing sequences of nucleic acid molecules in a sample provided herein further include applying a rule to compare non-identical sample barcodes at each end of the sequence read to allowed sample barcodes. In other aspects, methods for analyzing sequences of nucleic acid molecules in a sample provided herein further include applying one or more rules (1) to correct for errors within barcodes, (2) to correct for errors between barcodes at each end of a nucleic acid molecule, (3) for demultiplexing sequence reads into sample families, (4) for assigning sequence reads to molecular families, or any combination thereof. In some aspects, each oligonucleotide further includes non-barcode positions including sites for hybridization, sites for sequence primer binding, sites for amplification, or any combination thereof. In other aspects, methods for analyzing sequences of nucleic acid molecules in a sample provided herein further include use of a different genome with each oligonucleotide being tested to sensitively detect sequence read misassignment. In some aspects, methods for analyzing sequences of nucleic acid molecules in a sample provided herein further include storing nucleic acid sequence data without demultiplexing.

In one embodiment, the invention provides methods for labeling nucleic acid molecules in a sample including: attaching a plurality of oligonucleotides to the nucleic acid molecules including a barcode, each barcode including: (i) a sample barcode including a pre-determined number of sample index positions including one or more specific nucleotides, wherein the location of sample index positions varies between samples; and (ii) a molecular barcode including molecular index positions including a nucleotide that differs from the nucleotides at sample index positions, wherein sample index positions and molecular index positions are interspersed in a stretch of contiguous bases. In one aspect, the methods for labeling nucleic acid molecules in a sample provided herein can further include attaching an oligonucleotide including the same sample barcode to each end of a nucleic acid molecule. In some aspects, the pre-determined number of sample barcode positions varies among different sample barcodes. In other aspects, the barcode includes about 10 to about 35 nucleotides. In various aspects, the barcode includes about 12 to about 25 nucleotides. In some aspects, the sample barcode includes 2, 3, 4, 5, 6, 7, 8, 9, 10, 11, 12, 13, 14, 15, 16, 17, 18, 19, or 20 sample index positions. In other aspects, the sample barcode includes about 4 to about 12 sample index positions. In various aspects, the molecular barcode includes about 5 to about 25 molecular index positions. In some aspects, the molecular barcode includes about 5 to about 15 molecular index positions. In one aspect, sample index position nucleotides and molecular index position nucleotides are selected from: (A) the sample index position nucleotide is A and the molecular index position nucleotide is C, G, T, or a combination thereof (B) the sample index position nucleotide is T and the molecular index position nucleotide is C, G, A, or a combination thereof; (C) the sample index position nucleotide is C and the molecular index position nucleotide is G, A, T, or a combination thereof (D) the sample index position nucleotide is G and the molecular index position nucleotide is C, A, T, or a combination thereof (E) the sample index position nucleotide is A, T, or a combination thereof and the molecular index position nucleotide is C, G, or a combination thereof (F) the sample index position nucleotide is A, C, or a combination thereof and the molecular index position nucleotide is T, G, or a combination thereof; (G) the sample index position nucleotide is A, G, or a combination thereof and the molecular index position nucleotide is T, C, or a combination thereof; (H) the sample index position nucleotide is T, C, or a combination thereof and the molecular index position nucleotide is A, G, or a combination thereof (I) the sample index position nucleotide is T, G, or a combination thereof and the molecular index position nucleotide is A, C, or a combination thereof; or (J) the sample index position nucleotide is G, C, or a combination thereof and the molecular index position nucleotide is A, T, or a combination thereof. In some aspects, each barcode includes one or more additional index barcodes including index positions. In various aspects, the one or more additional barcode is a cellular barcode, a barcode that provides a measure of DNA length of an unrepaired end, or both a cellular barcode and a barcode that provides a measure of DNA length of an unrepaired end. In some aspects, each oligonucleotide further includes non-barcode positions including sites for hybridization, sites for sequence primer binding, sites for amplification, or any combination thereof. In other aspects, methods for labeling nucleic acid molecules in a sample provided herein can further include sequencing labeled nucleic acid molecules. In some aspects, sequencing labeled nucleic acid molecules further includes storing nucleic acid sequence data without demultiplexing. In various aspects, storing nucleic acid sequence data without demultiplexing prevents use of sequence data in the absence of a demultiplexing key and prevents unauthorized use of the data.

In another embodiment, the invention provides a method for identifying erroneous sequence reads including: (a) attaching a plurality of oligonucleotides to the nucleic acid molecules of the sample, wherein each oligonucleotide includes a barcode including: (i) a sample barcode including a pre-determined number of sample index positions including one or more specific nucleotides, wherein the location of sample index positions varies between samples, and wherein a same sample barcode is attached to each end of a nucleic acid molecule in the sample; and (ii) a molecular barcode including molecular index positions including a nucleotide that differs from the nucleotides at sample index positions, wherein sample index positions and molecular index positions are interspersed in a stretch of contiguous bases; and (b) sequencing the nucleic acid molecules, wherein sequence reads include barcode sequences, thereby identifying erroneous sequence reads.

In one aspect, identifying erroneous sequence reads includes identifying nucleic acid molecules with discrepant sample barcodes. In some aspects, sequencing errors are further corrected for by comparing sample barcodes at both ends of a sequence read. In other aspects, the nucleic acid molecules with discrepant sample barcodes are further removed from the sequence reads and/or from molecular families. In another aspect, identifying nucleic acid molecules with discrepant sample barcodes includes identifying misprimed nucleic acid molecules. In some aspects, misprimed nucleic acid molecules are corrected with proper barcodes and used for improving sequence quality. In other aspects, nucleic acid molecules with corrected barcodes are assigned to corrected read families. In various aspects, corrected read families are used to accurately determined distinct coverage. In some aspects, distinct coverage determination is used to evaluate libraries of nucleic acid molecules. In one aspect, the method further includes assigning the sequence reads to molecular families based on the location of molecular index positions and the nucleotide at each molecular index position. In some aspects, identifying erroneous sequence reads includes identifying nucleic acid molecules assigned to multiple molecular families. In other aspects, the nucleic acid molecules assigned to multiple molecular families are further removed from the sequence reads and/or from molecular families.

### BRIEF DESCRIPTION OF THE DRAWINGS

FIG. 1 shows a comparison of a traditional product barcode versus three floating DNA barcodes.

FIG. 2A shows 16 sample barcodes in digital format using 7/14 criteria.

FIG. 2B shows a conversion from digital to nucleotide format, 7/14 criteria.

FIG. 2C shows a conversion from degenerate to actual sequences for a single sample barcode, 7/20 bp format.

FIG. 3A shows standard barcodes.

FIG. 3B shows floating barcodes.

FIG. 4 shows generation of artifactual chimeric molecules with standard barcodes.

FIG. 5 shows alignment of human sequence reads to standard barcodes (left) and floating barcodes (right).

FIG. 6 shows the level of mispriming based on the abundance of adaptors in the ligation step.

FIG. 7 shows the ratio of mispriming rates i7:i5 based on the adapter concentration.

FIG. 8 shows the frequency of molecular barcode sequence repeats.

### DETAILED DESCRIPTION OF THE INVENTION

The present invention is based on the discovery that barcodes based on nucleotide location rather than sequence can be used to identify and group nucleic acid molecules and sequence reads.

Barcodes that are based on nucleotide location rather than sequence-based allow for flexibility in that a relatively low number of barcodes for one index and very high number of barcodes for another index or a high number of barcodes for two or more indices per barcode can be generated, for example. In addition, barcodes with pre-determined index positions allow for improved methods of error correction.

Systems and Sets of Oligonucleotides for Labeling Nucleic Acids

In one embodiment, the invention provides systems for labeling nucleic acid molecules in a sample including: a set of oligonucleotides including a plurality of barcodes, each barcode including a stretch of contiguous bases including: (i) a sample barcode including a pre-determined number of sample index positions including one or more specific nucleotides, wherein the location of sample index positions varies between samples; and (ii) a molecular barcode including molecular index positions including a nucleotide that differs from the nucleotide(s) at sample index positions, wherein molecular index positions are interspersed among sample index positions.

Systems for labeling nucleic acid molecules in a sample include sets of oligonucleotides. As used herein, “set of oligonucleotides” means a group or collection of oligonucleotides that can be used together. Accordingly, sets of oligonucleotides in the systems for labeling nucleic acid molecules in a sample provided herein can be used together to label nucleic acids. Subsets of sets of oligonucleotides can also be used in the systems for labeling nucleic acid molecules in a sample. As used herein, “subset of oligonucleotides” refers to only a portion or some of the oligonucleotides in a set of oligonucleotides for labeling nucleic acids in a sample. Accordingly, all or some of the oligonucleotides included in a set of oligonucleotides can be used for labeling nucleic acids in a sample.

As used herein, “labeling nucleic acid molecules” means modifying nucleic acid molecules for detection, identification, analysis, or purification, for example. In some aspects, nucleic acids are labeled by attaching one or more oligonucleotides to a nucleic acid molecule. An oligonucleotide can be attached to the end of a nucleic acid molecule. In some aspects, oligonucleotides are attached to both ends of a nucleic acid molecule. In other aspects, the oligonucleotides attached to the ends of a nucleic acid molecule differ in sequence. In some aspects, sample indices of oligonucleotides attached to the ends of a nucleic acid molecule are identical. In other aspects, molecular indices of oligonucleotides attached to the ends of a nucleic acid molecule differ.

Any nucleic acid molecule can be labeled, including DNA, RNA, and nucleic acid fragments, for example. DNA sources that can be labeled include, for example, chromosomal DNA, plasmid DNA, cDNA, cell-free DNA (cfDNA), circulating tumor DNA (ctDNA), and any fragment thereof. Labeled nucleic acids can be used for the preparation of nucleic acid libraries, for example. In some aspects, the library is a genomic library. Libraries including labeled nucleic acid molecules can be prepared by attaching sets or subsets of oligonucleotides provided herein to nucleic acid molecules through end-repair, A-tailing, and adapter ligation, for example. In some aspects, end repair and A-tailing is omitted and variable ends associated with a particular individual or set of indices included to determine the original end of a nucleic acid molecule, such as a DNA molecule, for example. Labeled nucleic acid molecules and libraries of labeled nucleic acid molecules can be analyzed by sequencing, for example. Any suitable sequencing method can be used to analyze labeled nucleic acid molecules.

Samples

Nucleic acids in a sample can be labeled using the systems for labeling nucleic acids and sets of oligonucleotides provided herein. Nucleic acids that can be labeled can be in any sample or any type of sample. In some aspects, the sample is blood, saliva, plasma, serum, urine, or other biological fluid. Additional exemplary biological fluids include serosal fluid, lymph, cerebrospinal fluid, mucosal secretion, vaginal fluid, ascites fluid, pleural fluid, pericardial fluid, peritoneal fluid, and abdominal fluid. In other aspects, the sample is a tissue sample. In some aspects, the sample is a cell sample or single cells. Fresh samples or stored samples can be used, including, for example, stored frozen samples, formalin-fixed paraffin-embedded (FFPE) samples, and samples preserved by any other method.

The sample can be from a normal or healthy subject. The sample can also be from a subject with a disease or disorder. Nucleic acids in a sample from a subject with any disease or disorder can be labeled using the systems and sets of oligonucleotides provided herein. In some aspects, the disease or disorder is cancer. In some aspects, the sample is a fluid sample from a subject with cancer. In other aspects, the sample is a tissue sample from a subject with cancer. In some aspects, the sample is a cell sample from a subject with cancer. In other aspects, the sample is a cancer sample. A cancer sample can be a sample from a solid tumor or a liquid tumor. The cancer can be kidney cancer, renal cancer, urinary bladder cancer, prostate cancer, uterine cancer, breast cancer, cervical cancer, ovarian cancer, lung cancer, colon cancer, rectal cancer, oral cavity cancer, pharynx cancer, pancreatic cancer, thyroid cancer, melanoma, skin cancer, head and neck cancer, brain cancer, hematopoietic cancer, leukemia, lymphoma, bone cancer, muscle cancer, sarcoma, rhabdomyosarcoma, and others.

Nucleic acids can be labeled in a sample. Nucleic acids can also be extracted, isolated, or purified from a sample prior to labeling. Any suitable method for extraction, isolation, or purification can be used. Exemplary methods include phenol-chloroform extraction, guanidinium-thiocyanate-phenol-chloroform extraction, gel purification, and use of columns and beads. Commercial kits can be used for extraction, isolation, or purification of nucleic acids.

Barcodes

Sets of oligonucleotides for labeling nucleic acid molecules in a sample provided herein can include a plurality of barcodes, each barcode including: (i) a sample barcode including a pre-determined number of sample index positions including one or more specific nucleotides, wherein the location of sample index positions varies between samples; and (ii) a molecular barcode including molecular index positions including a nucleotide that differs from the nucleotides at sample index positions, wherein sample index positions and molecular index positions are interspersed in a stretch of contiguous bases.

Barcode index positions can include a stretch of contiguous bases. As used herein, “contiguous bases” means bases are next to each other in a sequence. In some aspects, a stretch of contiguous bases can include barcode or index positions and non-barcode or non-index positions. In other aspects, a stretch of contiguous bases can include barcode or index positions and no non-barcode or non-index positions. In some aspects, the pre-determined number of sample barcode positions varies among different sample barcodes.

A barcode can include any number of nucleotides. As an example, a barcode can include about 10 to about 35 nucleotides. As another example, a barcode can include about 12 to about 25 nucleotides. As yet another example, a barcode can include about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, or more nucleotides. As yet another example, a barcode can include at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, or more nucleotides.

Index Positions

Barcodes provided herein can include one or more index positions. Exemplary index positions include sample index positions, molecular index positions, DNA end index positions, and cellular index positions. For example, barcodes can include sample index positions, DNA end index positions and molecular index positions. Barcodes can also include sample index positions, molecular index positions, cellular index positions, DNA end index positions, or any combination thereof.

As used herein, the term “index position” means a nucleotide position within a barcode that can be used to identify the origin or source of a nucleic acid molecule. Thus, index positions allow sequence reads generated from a nucleic acid molecule to be assigned to categories or groups based on origin or source of the nucleic acid molecule that gave rise to the sequence read. As an example, sample index positions can be used to identify the sample a nucleic acid molecule came from and allow for grouping of sequence reads generated from the nucleic acid molecule into sample categories. Accordingly, sequence reads generated from nucleic acid molecules from the same sample can be grouped together. As another example, molecular index positions can be used to identify a nucleic acid molecule that gave rise to a sequence read. Accordingly, molecular index positions can be used to group together sequence reads generated from the same nucleic acid molecule. As yet another example, cellular index positions can be used to identify the cell a nucleic acid molecule came from and allow for grouping of sequence reads generated from nucleic acid molecules into cell categories. Accordingly, sequence reads of nucleic acid molecules from the same cell can be grouped together.

DNA end index positions can signify the length of an unrepaired DNA end, for example. Oligonucleotides with different extensions can be prepared that are able to ligate with different DNA molecules that have not been repaired. Different length overhangs can be indexed to identify the length of the overhang that was present in the unrepaired DNA molecule. In some aspects, different length overhangs present in unrepaired DNA molecules are identified in cancer samples. In other aspects, different length overhangs present in unrepaired DNA molecules are identified to identify or detect cancer. Oligonucleotides can have any length of extension, including extensions of 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, or more. Oligonucleotides can also have 5′ or 3′ extensions.

Barcodes provided herein can include sample barcodes. A sample barcode can include a pre-determined number of sample index positions. As used herein, “pre-determined number of sample index positions” means that a particular number of positions can be assigned to a sample index to identify the sample a nucleic acid molecule came from. The number of pre-determined sample index positions can vary between samples. The location of sample index positions can also vary between samples. In some aspects, the number of pre-determined sample index positions and the location of sample index positions can vary between samples. Thus, a sample source for a nucleic acid molecule and sequence reads the nucleic acid molecules gave rise to can be identified by the number of sample index positions that form a sample barcode, the location of sample index positions, or both the number and location of sample index positions.

Because the location of sample index positions varies between samples in some embodiments, sample barcodes can be “floating” or “digital” barcodes. As used herein, “floating barcode” or “digital barcode” refers to a barcode with index positions whose location varies between groups or categories. Any barcode including index positions that can vary between groups or categories, such as sample barcodes including sample index positions, molecular barcodes including molecular index positions, cellular barcodes including cellular index positions, and others, can be a floating barcode. For example, in addition to the location of sample index positions that can vary, as described above, the location of molecular index positions of a molecular barcode can vary between different nucleic acid molecules that gave rise to sequence reads. As another example, the location of cellular index positions of a cellular barcode can vary between sequence reads obtained from nucleic acid molecules from different cells.

In some aspects, the pre-determined number of sample index positions in a sample barcode includes one or more specific nucleotides that define the type of index to which it corresponds. For example, the one or more specific nucleotide in a pre-determined number of sample index positions can be A, T, G, or C. As another example, the one or more specific nucleotides in a pre-determined number of sample index position can be A and T, A and C, A and G, T and C, T and G, or G and C.

In some aspects, sample barcodes include 2, 3, 4, 5, 6, 7, 8, 9, 10, 11, 12, 13, 14, 15, 16, 17, 18, 19, 20, or more sample index positions, or a combination thereof. In some aspects, sample barcodes include about 4 to about 12 sample index positions. In other aspects, sample barcodes include about 2, about 3, about 4, about 5, about 6, about 7, about 8, about 9, about 10, about 11, about 12, about 13, about 14, about 15, about 16, about 17, about 18, about 19, about 20, or more sample index positions, or a combination thereof. In some aspects, sample barcodes includes at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more sample index positions, or a combination thereof.

Barcodes provided herein can include molecular barcodes. Molecular barcodes can include molecular index positions that include a nucleotide(s) that differs from the nucleotides at sample index positions. For example, sample index position nucleotides and molecular index position nucleotides can be selected from: (A) the sample index position nucleotide is A and the molecular index position nucleotide is C, G, T, or a combination thereof; (B) the sample index position nucleotide is T and the molecular index position nucleotide is C, G, A, or a combination thereof; (C) the sample index position nucleotide is C and the molecular index position nucleotide is G, A, T, or a combination thereof; (D) the sample index position nucleotide is G and the molecular index position nucleotide is C, A, T, or a combination thereof; (E) the sample index position nucleotide is A, T, or a combination thereof and the molecular index position nucleotide is C, G, or a combination thereof; (F) the sample index position nucleotide is A, C, or a combination thereof and the molecular index position nucleotide is T, G, or a combination thereof; (G) the sample index position nucleotide is A, G, or a combination thereof and the molecular index position nucleotide is T, C, or a combination thereof; (H) the sample index position nucleotide is T, C, or a combination thereof and the molecular index position nucleotide is A, G, or a combination thereof; (I) the sample index position nucleotide is T, G, or a combination thereof and the molecular index position nucleotide is A, C, or a combination thereof; or (J) the sample index position nucleotide is G, C, or a combination thereof and the molecular index position nucleotide is A, T, or a combination thereof.

Sample index positions of the sample barcodes provided herein can be interspersed with molecular index positions. Thus, barcodes provided herein can include sample index positions and molecular index positions that need not be confined to a particular contiguous stretch or block of nucleotides. For example, not all sample index positions need to be next to each other, and not all molecular index positions need to be next to each other. Sample index positions and molecular index positions can alternate. Any number of molecular index positions can be in between sample index positions. Any number of molecular index positions can be in between any number of sample index positions. Any number of molecular index positions and any number of nucleotides that are not molecular index or other index positions can be in between sample index positions. Any number of molecular index positions and any number of nucleotides that are not molecular index or other index positions can be in between any number of sample index positions. Any number of nucleotides that are not sample index positions or molecular index positions can be in between sample index positions and molecular index positions.

Some sample index positions can be next to each other, while other sample index positions can be located next to any other nucleotide in a barcode that is not a sample index position. Sample index positions and molecular index position can be in any configuration that does not require all sample index positions to be next to each other, for example. Sample index positions and molecular index position can be in any configuration that does not require all molecular index positions to be next to each other, for example. Sample index positions and molecular index position can also be in any configuration that does not require all sample index positions and all molecular index positions to be next to each other, for example. Positions of any index barcode can be in any configuration that does not require all nucleotides of the index barcode to be next to each other. Exemplary barcode indices include sample barcodes, molecular barcodes, cellular barcodes, and others.

Molecular barcodes provided herein can include about 5 to about 25 molecular index positions. In some aspects, molecular barcodes provided herein include about 5 to about 15 molecular index positions. In other aspects, molecular barcodes provided herein include about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more, molecular index positions. In some aspects, molecular barcodes provided herein include at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, or more, molecular index positions. In some aspects, molecular barcodes provided herein include about 20 molecular index positions or fewer than about 20 molecular index positions.

A barcode provided herein can include one or more additional index barcodes including index positions. In some aspects, the one or more additional index barcode is a cellular barcode. Thus, barcodes provided herein can include sample barcodes, molecular barcodes, cellular barcodes, barcodes that provide a measure of unrepaired DNA end length, any other index barcode, or any combination thereof. Accordingly, barcodes provided herein can include sample index positions, molecular index positions, and any other index positions such as cellular index positions, for example, that are interspersed among each other. No index positions of the barcodes provided herein need to be confined to a particular contiguous stretch or block of nucleotides. Index barcodes and index positions can be in any configuration that does not require all index positions to be next to each other.

Each oligonucleotide in a set of oligonucleotides can further include non-barcode positions. Non-barcode positions included in an oligonucleotide can include sites for hybridization, sites for amplification, sites for sequence primer binding, and sites for hybridization, sequence primer binding, and amplification. Sites for hybridization, sequence primer binding, and sites for amplification can include about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides. Sites for hybridization can include sites for binding of probes, for example. Sites for amplification can include primer binding sites, for example. Sites for hybridization, sequence primer binding, and sites for amplification can be distinct from each other. Sites for hybridization, sequence primer binding, and sites for amplification can also overlap. Sites for hybridization, sequence primer binding, and sites for amplification can overlap to any extent. In some aspects, sites for hybridization, sequence primer binding, and sites for amplification overlap by about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides. In some aspects, sites for hybridization, sequence primer binding, and sites for amplification overlap completely. In other aspects, there is no overlap of sites for hybridization, sequence primer binding, and sites for amplification.

Methods for Analyzing Nucleic Acid Sequences

In another embodiment, the invention provides methods for analyzing sequences of nucleic acid molecules in a sample. Methods for analyzing nucleic acid sequences provided herein can include (a) attaching a plurality of oligonucleotides to nucleic acid molecules, wherein each oligonucleotide includes a barcode including: (i) a sample barcode including a pre-determined number of sample index positions including one or more specific nucleotides, wherein the location of sample index positions varies between samples; and (ii) a molecular barcode including molecular index positions including a nucleotide that differs from the nucleotides at sample index positions, wherein sample index positions and molecular index positions are interspersed in a stretch of contiguous bases; and (b) sequencing the nucleic acid molecules, wherein some sequence reads include barcode sequences.

Methods for analyzing nucleic acid sequences provided herein can include attaching a plurality of oligonucleotides to the nucleic acid molecules. The plurality of oligonucleotides that can be attached can include sets of oligonucleotides. In some aspects, the plurality of oligonucleotides that can be attached includes a subset of oligonucleotides. Any of the oligonucleotides provided herein, including sets and subsets of oligonucleotides, can be used in the methods for analyzing sequences of nucleic acid molecules or fragments thereof provided herein. Accordingly, each oligonucleotide of the plurality of oligonucleotides that can be attached can include a pre-determined number of sample index positions including one or more specific nucleotides. The location of the pre-determined number of sample index positions can vary between samples. Each oligonucleotide of the plurality of oligonucleotides can also include a molecular barcode including molecular index positions. Molecular index positions can include a nucleotide that differs from the nucleotides at sample index positions. Sample index positions and molecular index positions can be interspersed in a stretch of contiguous bases.

In other aspects, the methods for analyzing sequences of nucleic acid molecules provided herein include attaching an oligonucleotide including the same sample barcode to each end of a nucleic acid molecule. In some aspects, the pre-determined number of sample barcode positions varies among different sample barcodes. A stretch of contiguous identical bases can be absent in oligonucleotides including the same sample barcode because nucleotides included in a sample barcode can be interspersed with nucleotides included in a molecular barcode or constituting molecular index positions, nucleotides included in a cellular barcode or constituting cellular index positions, nucleotides included in any other index barcode or constituting any other index positions, nucleotides not included in an index barcode or not constituting index positions, or any combination thereof. Accordingly, in some aspects, oligonucleotides attached to each end of a nucleic acid molecule including the same sample barcode do not cross-hybridize and do not result in the generation of artifacts such as chimeric molecules during amplification, for example. In some aspects, methods for analyzing sequences of nucleic acid molecules provided herein include attaching an oligonucleotide including a different sample barcode to each end of a nucleic acid molecule.

In one aspect, methods for analyzing sequences of nucleic acid molecules provided herein include attaching an oligonucleotide including the same molecular barcode to each end of a nucleic acid molecule. A stretch of contiguous identical bases can be absent in oligonucleotides including the same molecular barcode because nucleotides included in a molecular barcode can be interspersed with nucleotides included in a sample barcode or constituting sample index positions, nucleotides included in a cellular barcode or constituting cellular index positions, nucleotides included in any other index barcode or constituting any other index positions, nucleotides not included in an index barcode or not constituting index positions, or any combination thereof. Accordingly, in some aspects, oligonucleotides attached to each end of a nucleic acid molecule including the same molecular barcode do not cross-hybridize and do not result in the generation of artifacts such as chimeric molecules during amplification, for example. In other aspects, the methods provided herein include attaching an oligonucleotide including a different molecular barcode to each end of a nucleic acid molecule.

In some aspects, methods for analyzing sequences of nucleic acid molecules provided herein include attaching an oligonucleotide including the same sample barcode and the same molecular barcode to each end of a nucleic acid molecule. A stretch of contiguous identical bases can be absent in oligonucleotides including the same sample barcode and the same molecular barcode because nucleotides included in a sample barcode and in a molecular barcode can be interspersed with nucleotides included in a cellular barcode or constituting cellular index positions, nucleotides included in any other index barcode or constituting any other index positions, nucleotides not included in an index barcode or not constituting index positions, or any combination thereof. Accordingly, in some aspects, oligonucleotides attached to each end of a nucleic acid molecule including the same sample barcode and the same molecular barcode do not cross-hybridize and do not result in the generation of artifacts such as chimeric molecules during amplification, for example. In other aspects, the methods provided herein include attaching an oligonucleotide including a different sample barcode and a different molecular barcode to each end of a nucleic acid molecule.

In some aspects, methods for analyzing sequences of nucleic acid molecules provided herein include attaching an oligonucleotide including the same sample barcode, the same molecular barcode, the same cellular barcode, the same barcode that provides a measure of unrepaired DNA end length, the same index barcode including any other index nucleotides, or any combination thereof, to each end of a nucleic acid molecule in the sample. A stretch of contiguous identical bases in a barcode including a sample barcode, a molecular barcode, a cellular barcode, nucleotides including any other index positions or index barcode, or any combination thereof can be absent because of interspersed nucleotides. Interspersed nucleotides can include nucleotides that are not included in an index barcode, do not constitute index positions, or nucleotides that are included in an index barcode or constitute index positions other than the index barcode or index positions the nucleotides are interspersed with. Thus, cross-hybridization and generation of artifacts such as chimeric molecules during amplification can be prevented. In one aspect, the methods provided herein include attaching an oligonucleotide including a different sample barcode, a different molecular barcode, a different cellular barcode, a different index barcode including any other index nucleotides, or any combination thereof, to each end of a nucleic acid molecule in the sample.

Any suitable method can be used for attaching an oligonucleotide including a barcode to an end of a nucleic acid molecule. In various aspects, the oligonucleotide is covalently attached.

Barcodes in the methods for analyzing sequences of nucleic acid molecules provided herein can include any number of nucleotides. As an example, a barcode in the methods for analyzing sequences of nucleic acid molecules provided herein can include about 10 to about 35 nucleotides. As another example, a barcode in the methods for analyzing sequences of nucleic acid molecules provided herein can include about 12 to about 25 nucleotides. As yet another example, a barcode in the methods for analyzing sequences of nucleic acid molecules provided herein can include about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, or more nucleotides. As yet another example, a barcode in the methods for analyzing sequences of nucleic acid molecules provided herein can include at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, or more nucleotides.

Barcodes in the methods for analyzing sequences of nucleic acid molecules provided herein can include one or more index positions. Exemplary index positions include sample index positions, molecular index positions, and cellular index positions. For example, barcodes in the methods for analyzing sequences of nucleic acid molecules provided herein can include sample index positions and molecular index positions. Barcodes in the methods for analyzing sequences of nucleic acid molecules provided herein can also include sample index positions, molecular index positions, cellular index positions, index positions that provide a measure of unrepaired DNA end length, or any combination thereof.

Barcodes in the methods for analyzing sequences of nucleic acid molecules provided herein can include sample barcodes. A sample barcode can include a pre-determined number of sample index positions. The number of pre-determined sample index positions can vary between samples. The location of sample index positions can also vary between samples. In some aspects, the number of pre-determined sample index positions and the location of sample index positions can vary between samples. Thus, a sample source for a nucleic acid molecule and sequence reads the nucleic acid molecules gave rise to can be identified by the number of sample index positions that form a sample barcode, the location of sample index positions, or both the number and location of sample index positions.

The pre-determined number of sample index positions in a sample barcode in the methods for analyzing sequences of nucleic acid molecules provided herein can include one or more specific nucleotides. For example, the one or more specific nucleotide in a pre-determined number of sample index positions can be A, T, G, or C. As another example, the one or more specific nucleotides in a pre-determined number of sample index position can be A and T, A and C, A and G, T and C, T and G, or G and C.

In some aspects, sample barcodes in the methods for analyzing sequences of nucleic acid molecules provided herein include 2, 3, 4, 5, 6, 7, 8, 9, 10, 11, 12, 13, 14, 15, 16, 17, 18, 19, 20, or more sample index positions, or a combination thereof. In some aspects, sample barcodes in the methods for analyzing sequences of nucleic acid molecules provided herein include about 4 to 12 sample index positions. In various aspects, sample barcodes in the methods for analyzing sequences of nucleic acid molecules provided herein include about 2, about 3, about 4, about 5, about 6, about 7, about 8, about 9, about 10, about 11, about 12, about 13, about 14, about 15, about 16, about 17, about 18, about 19, about 20, or more sample index positions, or a combination thereof. In one aspect, sample barcodes in the methods for analyzing sequences of nucleic acid molecules provided herein includes at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more sample index positions, or a combination thereof.

Barcodes in the methods for analyzing sequences of nucleic acid molecules provided herein can include molecular barcodes. Molecular barcodes in the methods for analyzing sequences of nucleic acid molecules provided herein can include molecular index positions that include a nucleotide that differs from the nucleotides at sample index positions. For example, sample index position nucleotides and molecular index position nucleotides can be selected from: (A) the sample index position nucleotide is A and the molecular index position nucleotide is C, G, T, or a combination thereof; (B) the sample index position nucleotide is T and the molecular index position nucleotide is C, G, A, or a combination thereof; (C) the sample index position nucleotide is C and the molecular index position nucleotide is G, A, T, or a combination thereof; (D) the sample index position nucleotide is G and the molecular index position nucleotide is C, A, T, or a combination thereof; (E) the sample index position nucleotide is A, T, or a combination thereof and the molecular index position nucleotide is C, G, or a combination thereof; (F) the sample index position nucleotide is A, C, or a combination thereof and the molecular index position nucleotide is T, G, or a combination thereof; (G) the sample index position nucleotide is A, G, or a combination thereof and the molecular index position nucleotide is T, C, or a combination thereof; (H) the sample index position nucleotide is T, C, or a combination thereof and the molecular index position nucleotide is A, G, or a combination thereof; (I) the sample index position nucleotide is T, G, or a combination thereof and the molecular index position nucleotide is A, C, or a combination thereof; or (J) the sample index position nucleotide is G, C, or a combination thereof and the molecular index position nucleotide is A, T, or a combination thereof.

Sample index positions of the sample barcodes in the methods for analyzing sequences of nucleic acid molecules provided herein can be interspersed with molecular index positions. Thus, barcodes in the methods for analyzing sequences of nucleic acid molecules provided herein can include sample index positions and molecular index positions that need not be confined to a particular contiguous stretch or block of nucleotides. For example, not all sample index positions need to be next to each other, and not all molecular index positions need to be next to each other. Sample index positions and molecular index positions can alternate. Any number of molecular index positions can be in between sample index positions. Any number of molecular index positions can be in between any number of sample index positions. Any number of molecular index positions and any number of nucleotides that are not molecular index or other index positions can be in between sample index positions. Any number of molecular index positions and any number of nucleotides that are not molecular index or other index positions can be in between any number of sample index positions. Any number of nucleotides that are not sample index positions or molecular index positions can be in between sample index positions and molecular index positions.

Some sample index positions can be next to each other, while other sample index positions can be located next to any other nucleotide in a barcode that is not a sample index position. Sample index positions and molecular index position can be in any configuration that does not require all sample index positions to be next to each other, for example. Sample index positions and molecular index position can be in any configuration that does not require all molecular index positions to be next to each other, for example. Sample index positions and molecular index position can also be in any configuration that does not require all sample index positions and all molecular index positions to be next to each other, for example. Positions of any index barcode can be in any configuration that does not require all nucleotides of the index barcode to be next to each other. Exemplary barcode indices include sample barcodes, molecular barcodes, cellular barcodes, and others.

Molecular barcodes in the methods for analyzing sequences of nucleic acid molecules provided herein can include about 5 to 25 molecular index positions. In one aspect, molecular barcodes in the methods for analyzing sequences of nucleic acid molecules provided herein include about 5 to about 15 molecular index positions. In some aspects, molecular barcodes in the methods for analyzing sequences of nucleic acid molecules provided herein include about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more, molecular index positions. In other aspects, molecular barcodes in the methods for analyzing sequences of nucleic acid molecules provided herein include at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, or more, molecular index positions.

Each barcode in the methods for analyzing sequences of nucleic acid molecules provided herein can include one or more additional index barcodes including index positions. In some aspects, the one or more additional index barcode is a cellular barcode. Thus, barcodes in the methods for analyzing sequences of nucleic acid molecules provided herein can include sample barcodes, molecular barcodes, cellular barcodes, any other index barcode, or any combination thereof. Accordingly, barcodes in the methods for analyzing sequences of nucleic acid molecules provided herein can include sample index positions, molecular index positions, and any other index positions such as cellular index positions, for example, that are interspersed among each other. No index positions of the barcodes provided herein need to be confined to a particular contiguous stretch or block of nucleotides. Index barcodes and index positions can be in any configuration that does not require all index positions to be next to each other.

Nucleic acid molecules with attached oligonucleotides provided herein can be analyzed by sequencing, for example. Sequence reads obtained can include barcode sequences. Any suitable sequencing method can be used to analyze nucleic acid molecules. Exemplary sequencing methods include Next Generation Sequencing (NGS), for example. Exemplary NGS methodologies include the Roche 454 sequencer, Life Technologies SOLiD systems, the Life Technologies Ion Torrent, BGI/MGI systems, Genapsys systems, and Illumina systems such as the Illumina Genome Analyzer II, Illumina MiSeq, Illumina HiSeq, Illumina NextSeq, and Illumina NovaSeq instruments. Sequencing can be performed for deep coverage for each nucleotide, including, for example, at least 2× coverage, at least 10× coverage; at least 20× coverage; at least 30× coverage; at least 40× coverage; at least 50× coverage; at least 60× coverage; at least 70× coverage; at least 80× coverage; at least 90× coverage; at least 100× coverage; at least 200× coverage; at least 300× coverage; at least 400× coverage; at least 500× coverage; at least 600× coverage; at least 700× coverage; at least 800× coverage; at least 900× coverage; at least 1,000× coverage; at least 2,000× coverage; at least 3,000× coverage; at least 4,000× coverage; at least 5,000× coverage; at least 6,000× coverage; at least 7,000× coverage; at least 8,000× coverage; at least 9,000× coverage; at least 10,000× coverage; at least 15,000× coverage; at least 20,000× coverage; and any number or range in between.

In some aspects, sequencing includes whole genome sequencing. In various aspects, sequencing includes exome sequencing or targeted panels. As used herein, the term “exome sequencing” refers to sequencing all protein coding exons of genes in a genome. Exome sequencing can include target enrichment methods such as array-based capture and in-solution capture of nucleic acid, for example. Targeted panels include a subset of regions of interest and may include both protein coding and non-coding regions.

Sequences of nucleic acids in any sample or type of sample can be analyzed using the methods provided herein. In some aspects, the sample is blood, saliva, plasma, serum, urine, or other biological fluid. Additional exemplary biological fluids include serosal fluid, lymph, cerebrospinal fluid, mucosal secretion, vaginal fluid, ascites fluid, pleural fluid, pericardial fluid, peritoneal fluid, and abdominal fluid. In some aspects, the sample is a tissue sample. In other aspects, the sample is a cell sample. Fresh samples or stored samples can be used, including, for example, stored frozen samples, formalin-fixed paraffin-embedded (FFPE) samples, and samples preserved by any other method.

The sample can be from a normal or healthy subject. The sample can also be from a subject with a disease or disorder. Sequences of nucleic acids in a sample from a subject with any disease or disorder can be analyzed using the methods provided herein. In some aspects, the disease or disorder is cancer. In other aspects, the sample is a fluid sample from a subject with cancer. In some aspects, the sample is a tissue sample from a subject with cancer. In other aspects, the sample is a cell sample from a subject with cancer. In some aspects, the sample is a cancer sample. A cancer sample can be a sample from a solid tumor or a liquid tumor. The cancer can be kidney cancer, renal cancer, urinary bladder cancer, prostate cancer, uterine cancer, breast cancer, cervical cancer, ovarian cancer, lung cancer, colon cancer, rectal cancer, oral cavity cancer, pharynx cancer, pancreatic cancer, thyroid cancer, melanoma, skin cancer, head and neck cancer, brain cancer, hematopoietic cancer, leukemia, lymphoma, bone cancer, muscle cancer, sarcoma, rhabdomyosarcoma, and others.

Nucleic acids can be extracted, isolated, or purified from a sample prior to sequencing. Any suitable method for extraction, isolation, or purification can be used. Exemplary methods include phenol-chloroform extraction, guanidinium-thiocyanate-phenol-chloroform extraction, gel purification, and use of columns and beads. Commercial kits can be used for extraction, isolation, or purification of nucleic acids.

Methods for analyzing sequences of nucleic acid molecules provided herein can include sequencing libraries of nucleic acid molecules. Libraries of nucleic acid molecules with attached oligonucleotides provided herein can be prepared. In some aspects, a genomic library is prepared. In some aspects, libraries of nucleic acid molecules or fragments thereof with attached oligonucleotides including barcodes provided herein are prepared by amplification. Nucleic acid molecules and fragments of nucleic acid molecules including attached oligonucleotides including barcodes provided herein can be amplified by polymerase chain reaction (PCR). Amplicons of nucleic acid molecules and fragments of nucleic acid molecules including attached oligonucleotides including barcodes provided herein can be sequenced. Any suitable sequencing method can be used to sequence nucleic acid molecules and fragments of nucleic acid molecules with attached oligonucleotides including barcodes provided herein.

Methods for analyzing sequences of nucleic acid molecules in a sample provided herein can further include assigning sequence reads to groups or categories. For example, sequence reads can be assigned to sample families based on the location and number of sample index positions. Accordingly, nucleic acid molecules giving rise to sequence reads can be assigned to the sample the nucleic acid molecules originated from. In some aspects, the number of sample index positions can be used for error correction. Sequence reads can also be assigned to molecular families based on the location of molecular index positions and the nucleotide at each molecular index position. The number and location of molecular index positions can also be used to assign sequence reads to molecular families. Thus, sequence reads can be assigned to a nucleic acid molecule that gave rise to the sequence reads. In some aspects, the number of molecular index positions can be used for error correction. As yet another example, sequence reads can be assigned to cellular families based on cellular index positions, such as location, number, and nucleotide at each cellular index position, and combinations thereof. Accordingly, sequence reads and nucleic acid molecules that gave rise to sequence reads can be assigned to a cell of origin. In one aspect, the number of cellular index positions can be used for error correction. Any assignment of sequence reads can be made according to index positions included in barcodes of oligonucleotides and sets of oligonucleotides provided herein.

Methods for analyzing sequences of nucleic acid molecules in a sample provided herein can further include correcting for sequencing errors. Sources of errors can include synthetic errors, sequencing artifacts or polymerase slippage during an amplification step, for example. Sequencing errors can be corrected by comparing the number and location of sample index positions in a sequence read to the pre-determined number and location of sample index positions.

Sequencing errors can also be corrected by comparing sample barcodes at both ends of a sequence read. A rule can be applied to compare non-identical sample barcodes at each end of a sequence read to allowed sample barcodes. In one aspect, a rule can be applied to compare non-identical sample barcodes at both ends of a sequencing read where oligonucleotides including identical sample barcodes are attached to each end of a nucleic acid molecule or a fragment thereof. In some aspects, a rule can be applied to compare non-identical sample barcodes at both ends of a sequencing read where oligonucleotides including non-identical sample barcodes are attached to each end of a nucleic acid molecule or a fragment thereof. In other aspect, methods for analyzing sequences of nucleic acid molecules provided herein include use of a different genome with each oligonucleotide being tested to sensitively detect read misassignment.

Methods for analyzing sequences of nucleic acid molecules in a sample can further include applying one or more rules (1) to correct for errors within barcodes, (2) to correct for errors between barcodes at each end of a nucleic acid molecule, (3) for demultiplexing sequence reads into sample families, (4) for assigning sequence reads to molecular families, or any combination thereof. As used herein, “demultiplexing” means assigning sequence reads to groups or categories such as sample families or a sample of origin where multiple samples have been pooled for sequencing, for example, molecular families, cellular families, or any other desired group or combinations of groups.

Each oligonucleotide in a set of oligonucleotides in the methods for analyzing sequences of nucleic acid molecules in a sample provided herein can further include non-barcode positions. Non-barcode positions included in an oligonucleotide can include sites for hybridization, sites for amplification, sites for sequence primer binding, and sites for hybridization, sequence primer binding, and amplification. Sites for hybridization, sequence primer binding, and sites for amplification can include about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides. Sites for hybridization can include sites for binding of probes, for example. Sites for amplification can include primer binding sites, for example. Sites for hybridization, sequence primer binding, and sites for amplification can be distinct from each other. Sites for hybridization, sequence primer binding, and sites for amplification can also overlap. Sites for hybridization, sequence primer binding, and sites for amplification can overlap to any extent. In some aspects, sites for hybridization, sequence primer binding, and sites for amplification overlap by about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides. In other aspects, sites for hybridization, sequence primer binding, and sites for amplification overlap completely. In one aspect, there is no overlap of sites for hybridization, sequence primer binding, and sites for amplification.

Methods for analyzing sequences of nucleic acid provided herein can further include storing nucleic acid sequence data without demultiplexing. A demultiplexing key can be used to assign sequence data to groups of sequencing reads, for example. Storing nucleic acid sequence data without demultiplexing can protect sequence data. For example, storing nucleic acid sequence data can prevent use of sequence data by individuals who do not possess a correct demultiplexing key, thereby preventing unauthorized use of the data.

Methods for Labeling Nucleic Acid Molecules

In one embodiment, the invention provides methods for labeling nucleic acid molecules in a sample including: attaching a plurality of oligonucleotides to the nucleic acid molecules including a barcode, each barcode including: (i) a sample barcode including a pre-determined number of sample index positions including one or more specific nucleotides, wherein the location of sample index positions varies between samples; and (ii) a molecular barcode including molecular index positions including a nucleotide that differs from the nucleotides at sample index positions, wherein sample index positions and molecular index positions are interspersed in a stretch of contiguous bases.

Any of the oligonucleotides provided herein, including sets and subsets of oligonucleotides, can be used to label nucleic acid molecules or fragments thereof in the methods for labeling nucleic acid molecules provided herein. In one aspect, the methods provided herein include attaching an oligonucleotide including the same sample barcode to each end of a nucleic acid molecule. In some aspects, the methods provided herein include attaching an oligonucleotide including a different sample barcode to each end of a nucleic acid molecule. In other aspects, the pre-determined number of sample barcode positions varies among different sample barcodes.

Any suitable method can be used for attaching an oligonucleotide including one or more barcodes to the end of a nucleic acid molecule. In some aspects, the oligonucleotide is covalently attached.

Nucleic acids in any sample can be labeled using the methods provided herein. Nucleic acids that can be labeled can be in any sample or any type of sample. In some aspects, the sample is blood, saliva, plasma, serum, urine, or other biological fluid. Additional exemplary biological fluids include serosal fluid, lymph, cerebrospinal fluid, mucosal secretion, vaginal fluid, ascites fluid, pleural fluid, pericardial fluid, peritoneal fluid, and abdominal fluid. In some aspects, the sample is a tissue sample. In other aspects, the sample is a cell sample. Fresh samples or stored samples can be used, including, for example, stored frozen samples, formalin-fixed paraffin-embedded (FFPE) samples, and samples preserved by any other method.

The sample can be from a normal or healthy subject. The sample can also be from a subject with a disease or disorder. Nucleic acids in a sample from a subject with any disease or disorder can be labeled using the methods provided herein. In one aspect, the disease or disorder is cancer. In some aspects, the sample is a fluid sample from a subject with cancer. In other aspects, the sample is a tissue sample from a subject with cancer. In some aspects, the sample is a cell sample from a subject with cancer. In other aspects, the sample is a cancer sample. A cancer sample can be a sample from a solid tumor or a liquid tumor. The cancer can be kidney cancer, renal cancer, urinary bladder cancer, prostate cancer, uterine cancer, breast cancer, cervical cancer, ovarian cancer, lung cancer, colon cancer, rectal cancer, oral cavity cancer, pharynx cancer, pancreatic cancer, thyroid cancer, melanoma, skin cancer, head and neck cancer, brain cancer, hematopoietic cancer, leukemia, lymphoma, bone cancer, muscle cancer, sarcoma, rhabdomyosarcoma, and others.

Nucleic acids can be labeled in a sample. Nucleic acids can also be extracted, isolated, or purified from a sample prior to labeling. Any suitable method for extraction, isolation, or purification can be used. Exemplary methods include phenol-chloroform extraction, guanidinium-thiocyanate-phenol-chloroform extraction, gel purification, and use of columns and beads. Commercial kits can be used for extraction, isolation, or purification of nucleic acids.

Labeled nucleic acids can be used for the preparation of nucleic acid libraries, for example. In some aspects, the library is a genomic library. Libraries including labeled nucleic acid molecules can be prepared by attaching sets or subsets of oligonucleotides provided herein to nucleic acid molecules or fragments thereof through end-repair, A-tailing, and adapter ligation, for example. In some aspects, end repair and A-tailing is omitted and variable ends associated with a particular individual or set of indices included to determine the original end of a nucleic acid molecule, such as a DNA molecule, for example. Labeled nucleic acid molecules and fragments thereof and libraries of labeled nucleic acid molecules and fragments thereof can be analyzed by sequencing, for example. Any suitable sequencing method can be used to analyze labeled nucleic acid molecules. Sequencing methods can further include storing nucleic acid sequence data without demultiplexing. A demultiplexing key can be used to assign sequence data to groups of sequencing reads, for example. Storing nucleic acid sequence data without demultiplexing can protect sequence data. For example, storing nucleic acid sequence data can prevent use of sequence data by individuals who do not possess a correct demultiplexing key, thereby preventing unauthorized use of the data.

A barcode in the methods for labeling nucleic acid molecules provided herein can include any number of nucleotides. As an example, a barcode can include about 10 to about 35 nucleotides. As another example, a barcode can include about 12 to about 25 nucleotides. As yet another example, a barcode can include about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, or more nucleotides. As yet another example, a barcode can include at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, or more nucleotides.

Barcodes in the methods for labeling nucleic acid molecules provided herein can include one or more index positions. Exemplary index positions include sample index positions, molecular index positions, DNA end index positions, and cellular index positions. For example, barcodes can include sample index positions and molecular index positions. Barcodes can also include sample index positions, molecular index positions, cellular index positions, DNA end index positions, or any combination thereof.

Barcodes in the methods for labeling nucleic acid molecules provided herein can include sample barcodes. A sample barcode can include a pre-determined number of sample index positions. The number of pre-determined sample index positions can vary between samples. The location of sample index positions can also vary between samples. In some aspects, the number of pre-determined sample index positions and the location of sample index positions can vary between samples. Thus, a sample source for a nucleic acid molecule and sequence reads the nucleic acid molecules gave rise to can be identified by the number of sample index positions that form a sample barcode, the location of sample index positions, or both the number and location of sample index positions.

The pre-determined number of sample index positions in a sample barcode in the methods for labeling nucleic acid molecules provided herein can include one or more specific nucleotides. For example, the one or more specific nucleotide in a pre-determined number of sample index positions can be A, T, G, or C. As another example, the one or more specific nucleotides in a pre-determined number of sample index position can be A and T, A and C, A and G, T and C, T and G, or G and C.

In some aspects, sample barcodes in the methods for labeling nucleic acid molecules provided herein include 2, 3, 4, 5, 6, 7, 8, 9, 10, 11, 12, 13, 14, 15, 16, 17, 18, 19, 20, or more sample index positions, or a combination thereof. In other aspects, sample barcodes in the methods for labeling nucleic acid molecules provided herein include about 4 to about 12 sample index positions. In some aspects, sample barcodes in the methods for labeling nucleic acid molecules provided herein include about 2, about 3, about 4, about 5, about 6, about 7, about 8, about 9, about 10, about 11, about 12, about 13, about 14, about 15, about 16, about 17, about 18, about 19, about 20, or more sample index positions, or a combination thereof. In other aspects, sample barcodes in the methods for labeling nucleic acid molecules provided herein include at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more sample index positions, or a combination thereof.

Barcodes in the methods for labeling nucleic acid molecules provided herein can include molecular barcodes. Molecular barcodes can include molecular index positions that include a nucleotide that differs from the nucleotides at sample index positions. For example, sample index position nucleotides and molecular index position nucleotides can be selected from: (A) the sample index position nucleotide is A and the molecular index position nucleotide is C, G, T, or a combination thereof; (B) the sample index position nucleotide is T and the molecular index position nucleotide is C, G, A, or a combination thereof; (C) the sample index position nucleotide is C and the molecular index position nucleotide is G, A, T, or a combination thereof; (D) the sample index position nucleotide is G and the molecular index position nucleotide is C, A, T, or a combination thereof; (E) the sample index position nucleotide is A, T, or a combination thereof and the molecular index position nucleotide is C, G, or a combination thereof; (F) the sample index position nucleotide is A, C, or a combination thereof and the molecular index position nucleotide is T, G, or a combination thereof; (G) the sample index position nucleotide is A, G, or a combination thereof and the molecular index position nucleotide is T, C, or a combination thereof; (H) the sample index position nucleotide is T, C, or a combination thereof and the molecular index position nucleotide is A, G, or a combination thereof; (I) the sample index position nucleotide is T, G, or a combination thereof and the molecular index position nucleotide is A, C, or a combination thereof; or (J) the sample index position nucleotide is G, C, or a combination thereof and the molecular index position nucleotide is A, T, or a combination thereof.

Sample index positions of the sample barcodes in the methods for labeling nucleic acid molecules provided herein can be interspersed with molecular index positions. Thus, barcodes in the methods for labeling nucleic acid molecules provided herein can include sample index positions and molecular index positions that need not be confined to a particular contiguous stretch or block of nucleotides. For example, not all sample index positions need to be next to each other, and not all molecular index positions need to be next to each other. Sample index positions and molecular index positions can alternate. Any number of molecular index positions can be in between sample index positions. Any number of molecular index positions can be in between any number of sample index positions. Any number of molecular index positions and any number of nucleotides that are not molecular index or other index positions can be in between sample index positions. Any number of molecular index positions and any number of nucleotides that are not molecular index or other index positions can be in between any number of sample index positions. Any number of nucleotides that are not sample index positions or molecular index positions can be in between sample index positions and molecular index positions.

Some sample index positions can be next to each other, while other sample index positions can be located next to any other nucleotide in a barcode that is not a sample index position. Sample index positions and molecular index position can be in any configuration that does not require all sample index positions to be next to each other, for example. Sample index positions and molecular index position can be in any configuration that does not require all molecular index positions to be next to each other, for example. Sample index positions and molecular index position can also be in any configuration that does not require all sample index positions and all molecular index positions to be next to each other, for example. Positions of any index barcode can be in any configuration that does not require all nucleotides of the index barcode to be next to each other. Exemplary barcode indices include sample barcodes, molecular barcodes, cellular barcodes, DNA end index positions, and others.

Molecular barcodes in the methods for labeling nucleic acid molecules provided herein can include about 5 to about 25 molecular index positions. In some aspects, molecular barcodes in the methods for labeling nucleic acid molecules provided herein include about 5 to about 15 molecular index positions. In other aspects, molecular barcodes in the methods for labeling nucleic acid molecules provided herein include about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more, molecular index positions. In various aspects, molecular barcodes in the methods for labeling nucleic acid molecules provided herein include at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, or more, molecular index positions.

A barcode in the methods for labeling nucleic acid molecules provided herein can include one or more additional index barcodes including index positions. In some aspects, the one or more additional index barcode is a cellular barcode. In other aspects, the one or more additional index barcode is a barcode that provides a measure or unrepaired DNA end length. Thus, barcodes in the methods for labeling nucleic acid molecules provided herein can include sample barcodes, molecular barcodes, cellular barcodes, barcodes providing a measure of unrepaired DNA end length, any other index barcode, or any combination thereof.

Accordingly, barcodes in the methods for labeling nucleic acid molecules provided herein can include sample index positions, molecular index positions, and any other index positions such as cellular index positions, for example, that are interspersed among each other. No index positions of the barcodes in the methods for labeling nucleic acid molecules provided herein need to be confined to a particular contiguous stretch or block of nucleotides. Index barcodes and index positions can be in any configuration that does not require all index positions to be next to each other.

Each oligonucleotide in a set of oligonucleotides in the methods for labeling nucleic acid molecules in a sample provided herein can further include non-barcode positions. Non-barcode positions included in an oligonucleotide can include sites for hybridization, sites for amplification, sites for sequence primer binding, and sites for hybridization, sequence primer binding, and amplification. Sites for hybridization, sequence primer binding, and sites for amplification can include about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides. Sites for hybridization can include sites for binding of probes, for example. Sites for amplification can include primer binding sites, for example. Sites for hybridization, sequence primer binding, and sites for amplification can be distinct from each other. Sites for hybridization, sequence primer binding, and sites for amplification can also overlap. Sites for hybridization, sequence primer binding, and sites for amplification can overlap to any extent. In some aspects, sites for hybridization, sequence primer binding, and sites for amplification overlap by about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides. In some aspects, sites for hybridization, sequence primer binding, and sites for amplification overlap completely. In other aspects, there is no overlap of sites for hybridization, sequence primer binding, and sites for amplification.

Methods for Identifying Erroneous Sequence Reads

In one embodiment, the invention provides a method for identifying erroneous sequence reads including: (a) attaching a plurality of oligonucleotides to the nucleic acid molecules of the sample, wherein each oligonucleotide includes a barcode including: (i) a sample barcode including a pre-determined number of sample index positions including one or more specific nucleotides, wherein the location of sample index positions varies between samples, and wherein a same sample barcode is attached to each end of a nucleic acid molecule in the sample; and (ii) a molecular barcode including molecular index positions including a nucleotide that differs from the nucleotides at sample index positions, wherein sample index positions and molecular index positions are interspersed in a stretch of contiguous bases; and (b) sequencing the nucleic acid molecules, wherein sequence reads include barcode sequences, thereby identifying erroneous sequence reads.

As used herein, the term “erroneous sequence read” is meant to refer to any sequencing error that can be identified by the methods described herein.

In one aspect, identifying erroneous sequence reads includes identifying nucleic acid molecules with discrepant sample barcodes.

The methods described herein rely on the attachment of a same sample barcode to each end of a nucleic acid molecule. The term “discrepant sample barcodes” refers to cases where, as a result of an error occurring during the preparation of the nucleic acid for sequencing, a nucleic acid molecule is attached to a barcode that is different at each end of the nucleic acid molecule. This may result in an erroneous assignment in molecular families, which can then interfere with the proper analysis of the sequence read.

In some aspect, sequencing errors are further corrected for by comparing sample barcodes at both ends of a sequence read. In other aspects, the nucleic acid molecules with discrepant sample barcodes are further removed from the sequence reads and/or from molecular families.

In another aspect, identifying nucleic acid molecules with discrepant sample barcodes includes identifying misprimed nucleic acid molecules.

As used herein a “misprimed nucleic acid molecule” can refer to a nucleic acid molecule that contain multiple pairs of molecular barcodes. In such case, the number of molecules can be wrongly inflated, and/or the wrong sample can be assigned to an incorrect molecular read, which can negatively impact the frequency and/or identity of read variants. Both cases lead to issues in the analysis and the clinical interpretation of the results.

In some aspects, misprimed nucleic acid molecules are corrected with proper barcodes and used for improving sequence quality. In other aspects, nucleic acid molecules with corrected barcodes are assigned to corrected read families.

In various aspects, corrected read families are used to accurately determine distinct coverage. In some aspects, distinct coverage determination is used to evaluate libraries of nucleic acid molecules.

In one aspect, the method further includes assigning the sequence reads to molecular families based on the location of molecular index positions and the nucleotide at each molecular index position. In some aspects, identifying erroneous sequence reads includes identifying nucleic acid molecules assigned to multiple molecular families. In other aspects, the nucleic acid molecules assigned to multiple molecular families are further removed from the sequence reads and/or from molecular families.

As used herein, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as is commonly understood by one of skill in the art to which this invention belongs.

“About” as used herein when referring to a measurable value such as an amount, a temporal duration, and the like, is meant to encompass variations of ±20% or ±10%, or ±5%, or even ±1% from the specified value, as such variations are appropriate for the disclosed compositions or to perform the disclosed methods.

As used herein, the term “nucleic acid” refers to any deoxyribonucleic acid (DNA) molecule, ribonucleic acid (RNA) molecule, or nucleic acid analogues. A DNA or RNA molecule can be double-stranded or single-stranded and can be of any size. Exemplary nucleic acids include, but are not limited to, chromosomal DNA, plasmid DNA, cDNA, cell-free DNA (cfDNA), circulating tumor DNA (ctDNA), mRNA, tRNA, rRNA, siRNA, micro RNA (miRNA or miR), hnRNA. Exemplary nucleic analogues include peptide nucleic acid, morpholino- and locked nucleic acid, glycol nucleic acid, and threose nucleic acid. As used herein, the term “nucleic acid molecule” is meant to include fragments of nucleic acid molecules as well as any full-length or non-fragmented nucleic acid molecule, for example.

As used herein, the term “nucleotide” includes both individual units of ribonucleic acid and deoxyribonucleic acid as well as nucleoside and nucleotide analogs, and modified nucleotides such as labeled nucleotides. In addition, “nucleotide” includes non-naturally occurring analogue structures, such as those in which the sugar, phosphate, and/or base units are absent or replaced by other chemical structures. Thus, the term “nucleotide” encompasses individual peptide nucleic acid (PNA) (Nielsen et al., Bioconjug. Chem. 1994; 5(1):3-7) and locked nucleic acid (LNA) (Braasch and Corey, Chem. Biol. 2001; 8(1): 1-7) units as well as other like units.

As used herein, the term “subject” refers to any individual or patient on which the methods disclosed herein are performed. The term “subject” can be used interchangeably with the term “individual” or “patient.” The subject can be a human, although the subject may be an animal, as will be appreciated by those in the art. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject. The subject may also be a plant or micro-organism.

As used herein, the terms “treat,” “treatment,” “therapy,” “therapeutic,” and the like refer to obtaining a desired pharmacologic and/or physiologic effect, including, but not limited to, alleviating, delaying or slowing the progression, reducing the effects or symptoms, preventing onset, inhibiting, ameliorating the onset of a diseases or disorder, obtaining a beneficial or desired result with respect to a disease, disorder, or medical condition, such as a therapeutic benefit and/or a prophylactic benefit. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some cases, for prophylactic benefit, treatment is administered to a subject at risk of developing a particular disease, or to a subject reporting one or more of the physiological symptoms of a disease, even though a diagnosis of this disease may not have been made. The methods of the present disclosure may be used with any mammal or other animal. In some cases, treatment can result in a decrease or cessation of symptoms. A prophylactic effect includes delaying or eliminating the appearance of a disease or condition, delaying, or eliminating the onset of symptoms of a disease or condition, slowing, halting, or reversing the progression of a disease or condition, or any combination thereof.

### EXAMPLES

### Example 1

This example describes the design of floating/digital barcodes for multiply indexed samples.

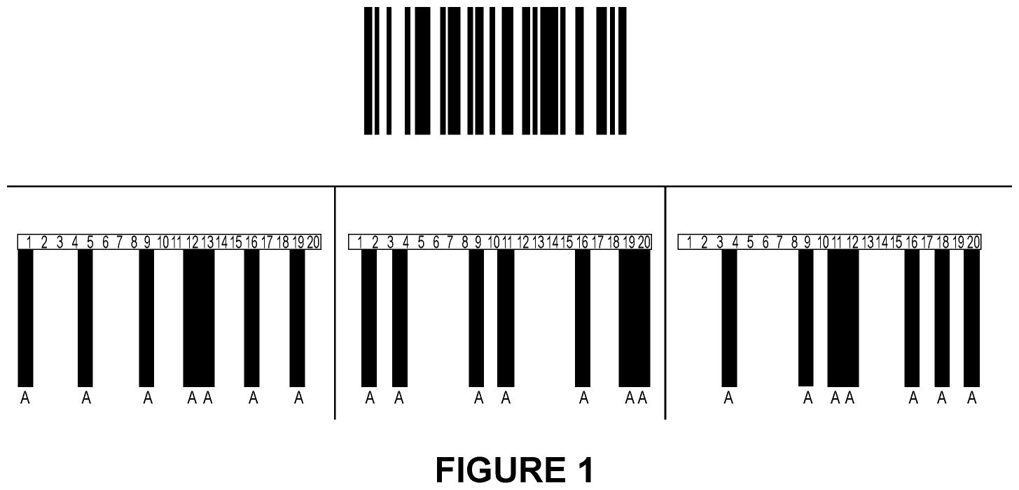

The presence or absence of a nucleotide at a given position of a floating or digital barcode provides information content, similar to a consumer product barcodes (UPCs) (FIG. 1). For different indices, the nucleotides or “bars” move or float to different positions and those new positions signify an alternate index. The number of possible barcodes increases rapidly as the sequence locations available increases. Positions not being used for the primary index can be used for secondary or additional indices. It is also possible to include additional levels of indexing that would be useful in methods such as single cell sequencing. For single cell sequencing, it would be possible to have a sample index, a cellular index, and a molecular index all within the single barcode, for example. Depending on the choice of conditions for creating barcodes, different numbers of primary and secondary barcodes are available, and the strength of error detection and error correction can be tuned as needed.

The number of different molecules in a sample is typically very high, with millions or more molecules being sequenced for each sample. With such a high number of molecules, it is generally not possible to synthesize and purify individual oligonucleotides for each molecular barcode. Degenerate nucleotides at multiple positions are often used to provide the diversity needed for distinguishing different molecules. Typically, the defined sample barcodes and the randomized molecular barcodes are segregated from each other for analysis. With a floating/digital barcode system, the multiple types of barcodes are intermingled within a region.

Compared to the standard fixed length barcodes, this represents a fundamentally different method for indexing samples that uses a location-based method where sequences are not directly compared to a reference. The location of the sample barcode varies with the sample and that location is used to identify sample families. With standard barcodes, sequences are compared to each other and perfect or near perfect sequence identities are grouped together as a sample family. With floating/digital barcodes, sequences are not directly compared to each other but rather are used to mark locations in a digital +/− manner. The +/− location data is then used to distinguish samples similar to a traditional product barcode (FIG. 1). In the example shown in FIG. 1, any position with the nucleotide “A” is part of the sample barcode while any other nucleotide is part of the molecular barcode. Whenever an “A” is sequenced, its location is noted and used for determining sample families.

The new type of barcode was designed based on multiple requirements, including the following, for example: (1) there should be enough unique barcodes to accommodate the number of samples and molecules on any run; (2) the combined sample/molecular barcodes on the different ends of each molecular read should be different but the sample barcode predictable in order to detect index hopping on high capacity sequencers; (3) barcodes should not contain extensive polynucleotide repeats or extremes in base composition that affect sequence quality; (4) molecular indices should be highly variable in order to distinguish all possible molecules; and (5) sample barcode design should be compatible with a viable number of oligonucleotide syntheses.

The novel design of a floating or digital barcode meets the criteria above. The novel barcode design is able to incorporate all these features within a relatively short sequence that is already compatible with both NextSeq and NovaSeq Illumina sequencers, for example. The same or similar designs can be made to be compatible with other sequencing systems.

The new floating/digital barcode intermingles sample and molecular barcodes at adjacent positions and uses location information rather than a direct sequence comparison to assign sample families. The nucleotide sequence at any given position is used to determine whether that position should be designated as a sample or molecular position. This location information is then used for determining the barcode and assigning sample families. If the number of sample barcode locations does not match the expected number or position, the molecule can either be discarded or attempts can be made to correct the barcode. The design of these barcodes allows flexible allotment of barcodes and classes such that it can be used in a variety of applications including multiplex samples on a sequencing run or single cell approaches in which reads need to assigned to a particular sample and cell.

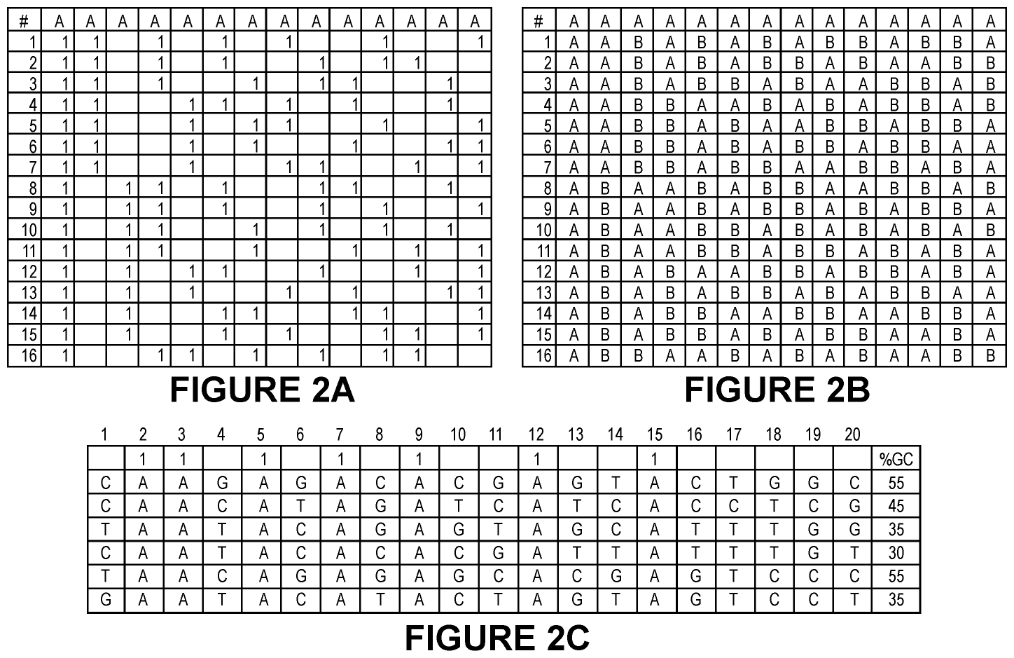

Many configurations of barcodes are possible. As one example of many possibilities, the sample index can always be the nucleotide “A” while the molecular index can be any of the other nucleotides (C, G, T). Using IUPAC nomenclature, C, G, or T is represented by the symbol “B” and A, C, or G is represented by the symbol “V.” Examples of sequences that could potentially be used in this fashion are shown in FIGS. 2A-2C.

The number of possible barcodes for a given number of positions (n) with can be calculated from the equation:

Cr=n!/r!(n−r)!

where n is the number of possible positions and r is the number of positions to be filled. The maximum number of possibilities for various sequence sizes is shown in Table 1.

TABLE 1
Possible Barcode Combinations

| Length of Mixed | Maximum # of |
|---|---|
| Sequence | Different Barcodes |
| 4 | 6 |
| 6 | 20 |
| 8 | 70 |
| 10 | 252 |
| 12 | 924 |
| 14 | 3432 |
| 16 | 12,870 |
| 18 | 48,620 |
| 20 | 184,756 |
| n | Cr = n!/r! (n − r)! |

At each position, a binary choice determines whether the position is used as a molecular index or sample index position. If the sequence matches the sample index sequence (e.g., A), it is part of the sample barcode. If it does not match (e.g., C, G, or T), it is part of the degenerate molecular index. In the example shown in FIG. 2C, within each 20 nt segment, up to 7 positions are allocated to sample index positions and 13 or more are three-fold degenerate making each sample barcode 20 nt stretch 3\hat{ } 13 or 1,594,323-fold degenerate. Because each molecule has two such barcodes, any individual molecule can be 1,594,323\hat{ }2 or 2.5 trillion-fold degenerate.

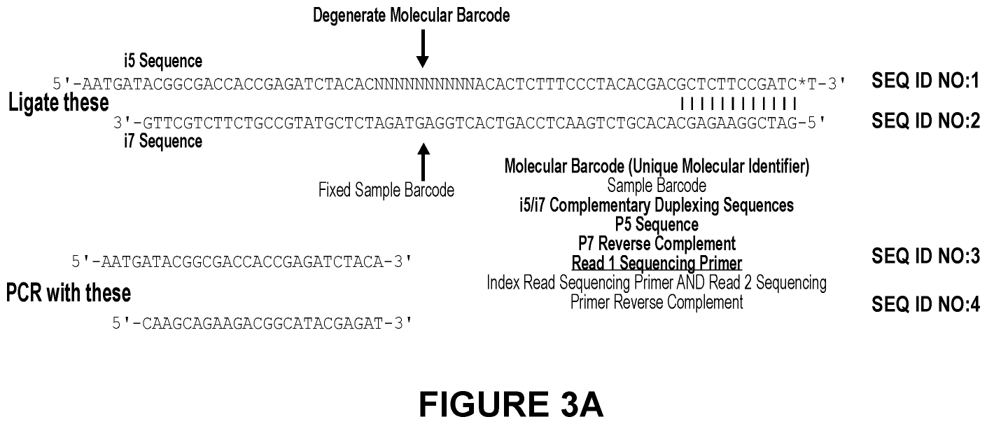

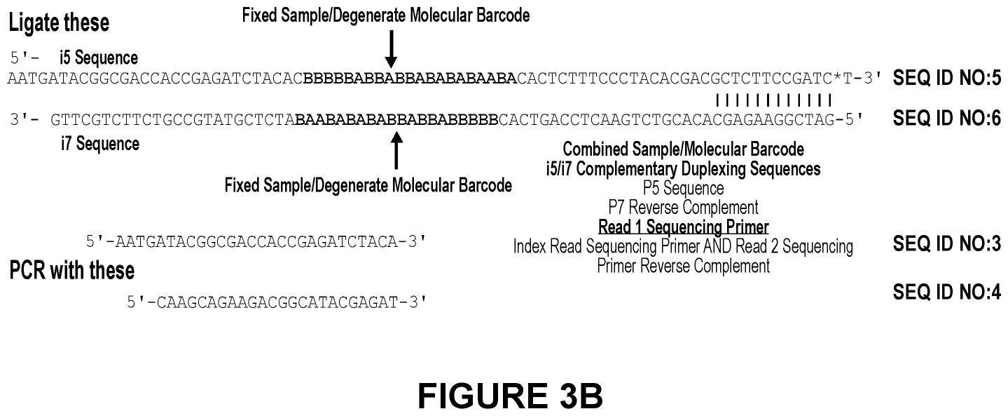

As shown in FIG. 3A, many types of standard adapters have the degenerate molecular barcode and the fixed sample barcode located on different adapter oligonucleotides (see SEQ ID NOs:1 and 2). This is not the case for floating barcodes where the two are intermingled as shown in FIG. 3B (see SEQ ID NOs:5 and 6).

Error correction and the pattern of sample and molecular barcodes can take a variety of forms. In some cases, such as sequencing of somatic variants, it is important that reads are not misassigned. Thus, having robust error detection and correction is important. For example, if there is a fixed number of sample barcode positions, matching that number provides one type of quality check. If the barcode is not the selected length, there must be a sequencing error in that particular molecule. It may be possible to correct the error based on the expected barcodes or it may require eliminating a sequence from the overall results in order to avoid misassignment. Alternatively, it is possible to use a variable number of sample barcode positions but generate them in such a way that any single sequencing error can be detected and fixed based on allowable patterns. In such cases, every sample barcode differs from all other sample barcodes by at least two or at least three or more changes. In other cases, occasional misassignment may not be a significant issue, with a higher importance placed on providing the maximal number of barcodes. This would prevent some types of error detection/correction but still allow comparison of barcodes at both ends of the same molecule.

In addition to a single nucleotide representing the sample barcode, other variations are possible. For example, the sample (or cellular) barcode could be represented by either a fixed A or T and the molecular barcode by degenerate G/C. This configuration generates many more sample/cellular barcodes with fewer molecular barcodes. Altering the number and degeneracy of the sample/molecular barcode positions allows one to optimize the number of both to the application at hand.

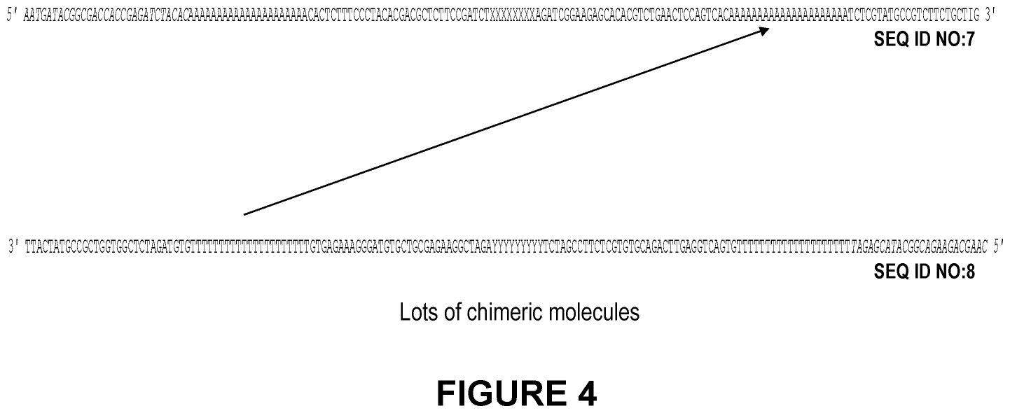

A floating or digital barcode system allows for the same sample barcode to be put at both ends of the same nucleic acid molecule. With traditional DNA barcodes, the same sample barcode cannot be used at both ends of the same molecule. If the identical standard sample barcode were placed at both ends of the same molecule, different molecules could cross-hybridize, resulting in a high risk of generating artifactual chimeric molecules during the amplification. With the same barcode sequence at both ends of a molecule, the two 3′ most regions could hybridize and generate a partially duplicated molecule. Since standard sample barcodes could be present millions of times in a sample being amplified, the potential for a chimeric molecule formation is high (see FIG. 4 and SEQ ID NOs:7 and 8). This is not the case for floating barcodes because, even with the same sample barcode, there is no long stretch of contiguous identical bases. Because the sample barcodes for floating adaptors have only short regions of homology, there is little risk of non-specific interactions and chimera formation. The same sample barcode can thus be placed at both ends of the same molecule, allowing for comparison of the two barcodes for errors in the other. If no errors are found, the sample can be confidently assigned. If the two barcodes are not identical, they can be compared to a list of allowed barcodes and corrected accordingly. The number of barcodes used for each index determines the degree to which errors can be corrected.

Thus, the ability to put the same sample barcode on both ends of the same molecule with low risk of chimera formation provides a simple but powerful error correction potential. One simply compares the sample barcodes at each end of the molecule to verify identity. If the same, the molecule can be placed in the proper sample family. If they do not match, both can be compared to an allowable set of sample barcodes and the errant barcode potentially corrected. This method provides a powerful way to ensure that molecules are assigned to the proper sample family with minimal loss of reads. An example of sample barcode correction is shown in Table 2. The edit distance between barcodes will determine how barcodes are corrected with greater ability to correct barcodes and retain reads when the edit distance is higher.

The lack of agreement of sample barcodes on the different ends of the same molecule provides evidence for problematic processes in sample preparation. By monitoring the frequency of chimeric molecules as evidenced by non-matching sample barcodes, improvements can be made in library preparation and sequencing methodologies.

If a specific molecular barcode is matched with multiple different molecular barcodes and the number of mismatches indicates it is not caused by a simple sequencing error, it indicates that one or more molecular reads are mismatched. The relative frequency of molecular pairs can be used to determine which is the predominant species and can be used as is and which is likely to be an artifact and requires correction or removal. See Table 3 for the breakdown of how the i5 and i7 adaptors are distributed for one pair of samples. The correct and correctable barcodes can be used in a straightforward manner while the misprimed molecules require a more complex analysis if the read is to be salvaged. Without knowing which reads are misprimed, incorrect information could be incorporated into the analysis. Knowing where the mispriming has occurred allows the proper handling of the sequence reads. Mispriming can only be corrected when it is at a low enough level that it can be reliably detected.

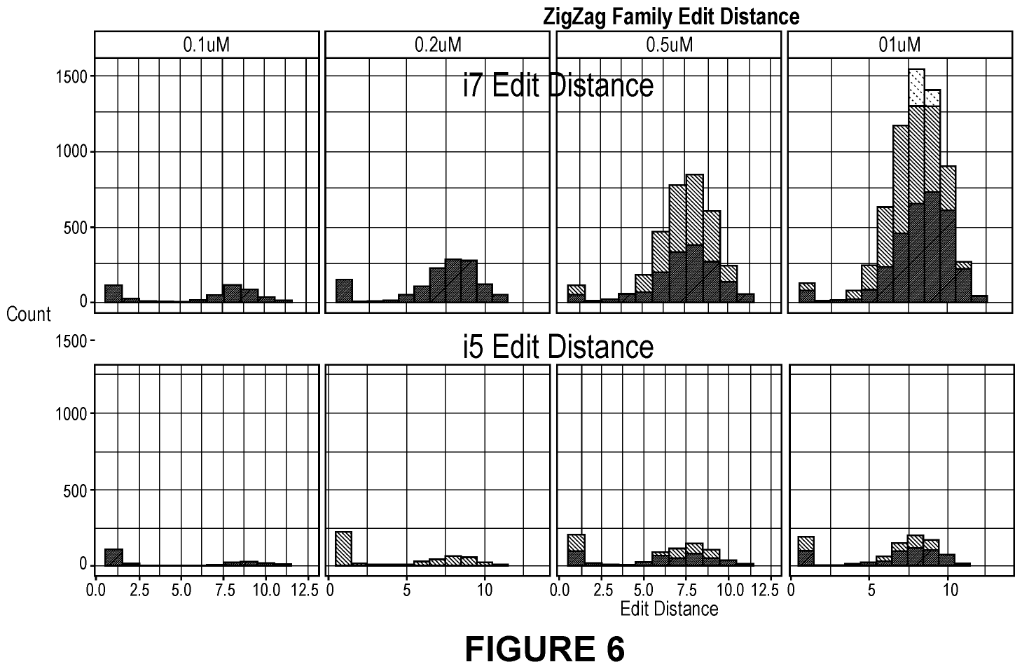

As shown in FIG. 6, an over-abundance of adaptors in the ligation step can lead to significant problems when residual adaptors are extended by PCR primers (e.g., SEQ ID NOs:3 and 4) and subsequently used in later stages of amplification. At 0.2 μM and below, there is a relatively low level of mispriming while it grows substantially at 0.5 μM and above.

TABLE 2
Correction of Sample Barcodes from Same
Molecule Reads with Edit Distance = 2

| | | Patterns | Fragment |
|---|---|---|---|
| i7 distance | i5 distance | match? | Assignment |
| 0 | 0 | Yes | i7/i5 |
| 1 | 0 | Yes | i7/i5 |
| 1 | 1 | No | none |
| n/a | 0 | n/a | i5 |
| n/a | 1 | n/a | none |
| 0 | 1 | No | none |
| n/a | n/a | n/a | none |

TABLE 3
Distribution of i5 and i7 adaptors for one pair of samples

| | edit distance | Status | Sample 1 | Sample 2 |
|---|---|---|---|---|
| i7 | 0 | Correct | 13.3% | 10.5% |
| | 1, 2, 3 | Correctable | 1.5% | 0.8% |
| | >3 | Mispriming error | 85.2% | 88.7% |
| i5 | 0 | Correct | 90.5% | 87.8% |
| | 1, 2, 3 | Correctable | 0.5% | 1.0% |
| | >3 | Mispriming error | 9.0% | 11.2% |

In summary, the fundamental difference in the approach to design novel floating or digital barcodes was to use nucleotide locations as the barcode rather than a specific nucleotide sequence. There are multiple possible variations on this theme that allow for flexibility in the number of barcodes and methods of error correction. Some of the benefits of the new barcodes include (1) improved assignment of NGS reads to sample and molecular families; (2) reduction in the number of oligo synthesis/purification for complex samples; (3) reduction in the number of problematic homopolymers and GC-rich stretches in degenerate regions.

### Example 2

This example describes testing of floating barcodes with samples.

To test floating barcodes, an experiment was designed to detect read mismatches with maximal sensitivity. Standard library preparation protocols were used. No significant difference in yield was observed between standard and floating barcodes.

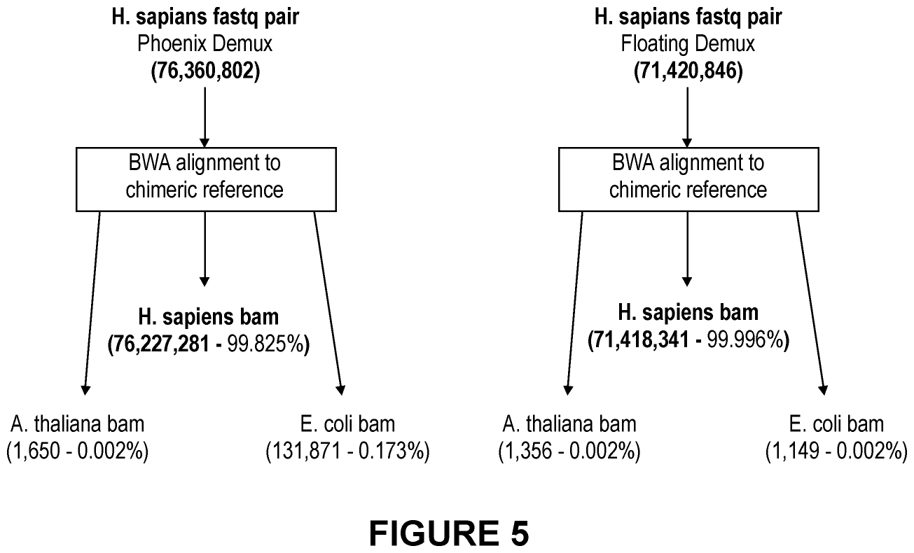

To detect misassignment, three samples were prepared and sequenced in parallel with both standard and floating barcodes. Each sample was prepared using a different barcode. The three samples were human DNA captured using a targeted panel for human DNA and genomic DNA from E. coli and Arabidopsis thaliana that had been sheared but not selectively captured. All six samples were run on the same NextSeq sequencing run set for 20nt index sequencing. The resulting reads were then demultiplexed twice, once using standard barcodes and once using floating barcodes. The reads were then separately analyzed to see to which genome reads aligned. With human aligned sequences, initial algorithms were as good or better than standard alignments, with less than 0.002% of reads aligning to barcodes assigned to E. coli and Arabidopsis thaliana as shown in FIG. 5. The lower off-target read mapping led to lower error rates for read assignments.

These data show that floating or digital barcodes performed well when compared to standard barcodes. Optimization of laboratory protocols, including altering blockers, for example, and software/algorithms, including software for demultiplexing, error correction, and creation of read families, for example, will further improve results obtained with floating or digital barcodes for sequence analysis. In addition, floating or digital barcodes can be used in a variety of applications where multiple indices are useful, such as marking cells in single-cell analysis and systems where one, two, three, or more indices are useful for marking molecular, cellular, and/or sample properties and grouping into the respective categories, for example.

In summary, the novel floating or digital barcode system provides multiple advantages for analysis, such as flexibility, lower cost of oligo synthesis, and easy methods for error correction that, unexpectedly and surprisingly, present an improvement over current methods of error correction, leading to better assignment of reads to the correct sample and molecular families, for example.

### Example 3

This example describes how floating barcodes can be used to identify and remove incorrectly assigned molecular reads from samples.

Because the sample barcode is encoded at both ends of each molecule, the barcodes can be compared both for error correction and confirmation that undesired, chimeric molecules arising from multiple samples have not occurred to a significant extent. As shown in FIG. 6, the formation of chimeric molecules can be a significant issue even using standard conditions. The problem can take the form of the same molecule acquiring multiple pairs of molecular barcodes and artifactually inflating the number of molecules or the wrong sample being assigned to a molecular read leading to incorrect frequency or identity of variants. Both situations lead to analysis issues that can affect clinical interpretation of results.

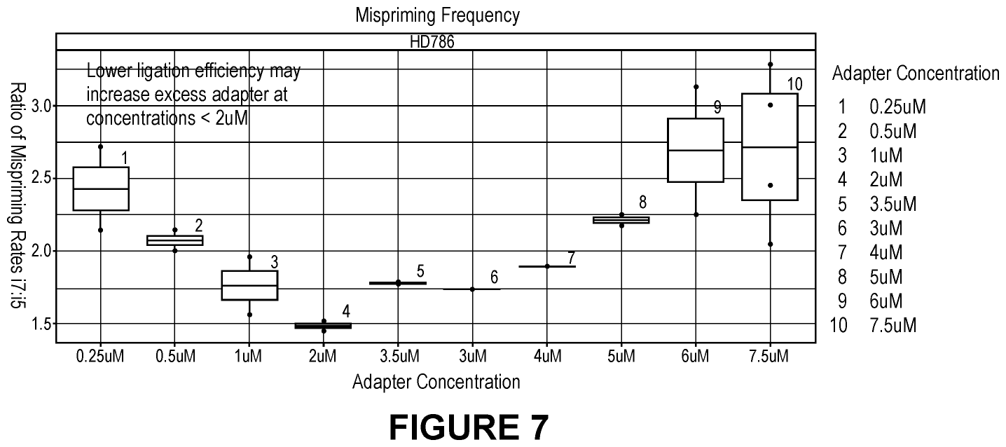

The absolute and relative concentrations of amplification primers in library preparation leads to variations in efficiency and accuracy of barcodes. The higher the initial concentration of adaptors, the more efficient the ligation and the greater fraction of a sample that can be recovered. Unfortunately, excess adaptors can lead to amplification issues with adaptors being amplified or used as primers with added barcodes being added during amplification rather than just the ligation stage (FIG. 7). If new sample barcodes are added during amplification, reads will be assigned to the wrong sample and the frequency or presence of variants becomes less accurate. If new molecular barcodes are added during amplification, each molecule has multiple pairs of barcodes so that molecular diversity will be overestimated, and error correction of those reads made more difficult or impossible. With standard barcodes, it is not even possible to measure the extent of these problems. With floating barcodes, such issues are readily detected, and methods can then be improved to optimize accuracy.

### Example 4

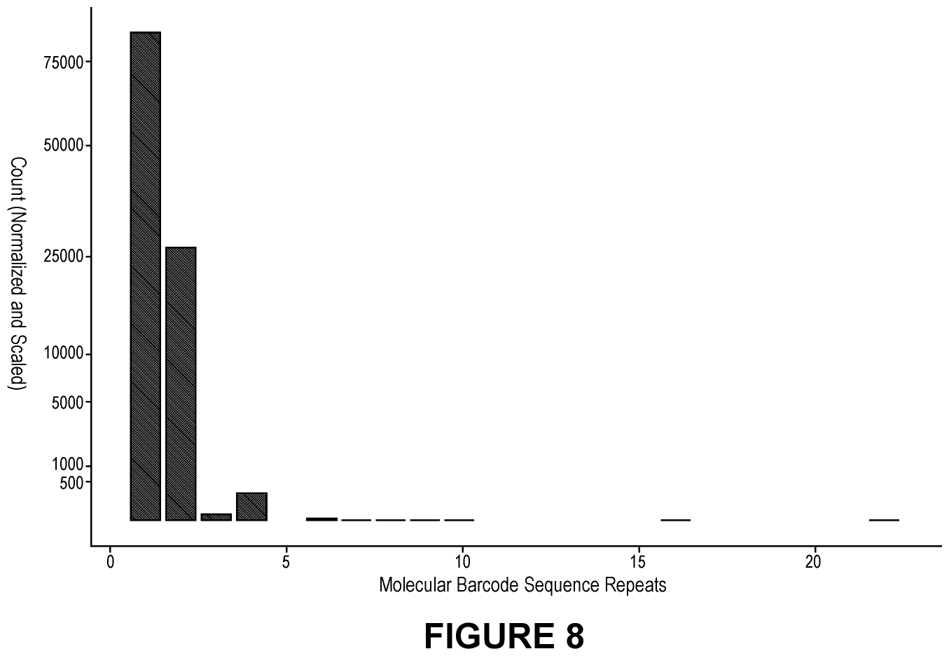

The molecular barcode is random but, because it is interspersed within the sample barcode, it does not contain long stretches of completely random bases that can cause problems. Completely random barcodes can be 100% GC while the 20 nt overall sequence must contain the sample barcode which can be all A or all T, thus setting an upper limit on GC content, typically 65%. This also prevents long homopolymers. Completely random barcodes have been shown to have certain sequences that can occur at hundreds of copies while most sequences occur only a few times. [Kinde I, Wu J, Papadopoulos N, Kinzler K W, Vogelstein B. Detection and quantification of rare mutations with massively parallel sequencing. Proc Natl Acad Sci USA. 2011 Jun. 7; 108(23):9530-5. doi: 10.1073/pnas.1105422108. Epub 2011 May 17. PMID: 21586637; PMCID: PMC3111315.] The more even content of these molecular barcodes is shown in FIG. 8 where few barcodes are significantly over-represented.

Although the invention has been described with reference to the above examples, it will be understood that modifications and variations are encompassed within the spirit and scope of the invention. Accordingly, the invention is limited only by the following claims.

1. A system for labeling nucleic acid molecules in a sample comprising: a set of oligonucleotides comprising a plurality of barcodes, each barcode comprising a stretch of contiguous bases comprising:
(i) a sample barcode comprising a pre-determined number of sample index positions comprising one or more specific nucleotides, wherein the location of sample index positions varies between samples; and
(ii) a molecular barcode comprising molecular index positions comprising a nucleotide that differs from the nucleotides at sample index positions,
wherein sample index positions are interspersed among molecular index positions.

2. The system of claim 1, wherein the pre-determined number of sample barcode positions varies among different sample barcodes.

3. The system of claim 1, wherein the barcode comprises about 10 to about 35 nucleotides.

4. The system of claim 1, wherein the barcode comprises about 12 to about 25 nucleotides.

5. The system of claim 1, wherein the sample barcode comprises 2, 3, 4, 5, 6, 7, 8, 9, 10, 11, 12, 13, 14, 15, 16, 17, 18, 19, or 20 sample index positions, or a combination thereof.

6. The system of claim 1, wherein the sample barcode comprises about 4 to about 12 sample index positions.

7. The system of claim 1, wherein the molecular barcode comprises about 5 to about 25 molecular index positions.

8. The system of claim 1, wherein the molecular barcode comprises about 5 to about 15 molecular index positions.

9. The system of claim 1, wherein sample index position nucleotides and molecular index position nucleotides are selected from:
(A) the sample index position nucleotide is A and the molecular index position nucleotide is C, G, T, or a combination thereof;
(B) the sample index position nucleotide is T and the molecular index position nucleotide is C, G, A, or a combination thereof;
(C) the sample index position nucleotide is C and the molecular index position nucleotide is G, A, T, or a combination thereof;
(D) the sample index position nucleotide is G and the molecular index position nucleotide is C, A, T, or a combination thereof;
(E) the sample index position nucleotide is A, T, or a combination thereof and the molecular index position nucleotide is C, G, or a combination thereof;
(F) the sample index position nucleotide is A, C, or a combination thereof and the molecular index position nucleotide is T, G, or a combination thereof;
(G) the sample index position nucleotide is A, G, or a combination thereof and the molecular index position nucleotide is T, C, or a combination thereof;
(H) the sample index position nucleotide is T, C, or a combination thereof and the molecular index position nucleotide is A, G, or a combination thereof;
(I) the sample index position nucleotide is T, G, or a combination thereof and the molecular index position nucleotide is A, C, or a combination thereof; or
(J) the sample index position nucleotide is G, C, or a combination thereof and the molecular index position nucleotide is A, T, or a combination thereof.

10. The system of claim 1, wherein each barcode comprises one or more additional index barcodes comprising index positions.

11. The system of claim 10, wherein the one or more additional index barcode is a cellular barcode, a barcode that provides a measure of DNA length of an unrepaired end, or both a cellular barcode and a barcode that provides a measure of DNA length of an unrepaired end.

12. The system of claim 1, wherein each oligonucleotide in the set of oligonucleotides further comprises non-barcode positions comprising sites for hybridization, sites for sequence primer binding, sites for amplification, or any combination thereof.

13. A set of oligonucleotides for labeling nucleic acid molecules in a sample comprising a plurality of barcodes, each barcode comprising:
(i) a sample barcode comprising a pre-determined number of sample index positions comprising one or more specific nucleotides, wherein the location of sample index positions varies between samples; and
(ii) a molecular barcode comprising molecular index positions comprising a nucleotide that differs from the nucleotides at sample index positions,
wherein sample index positions and molecular index positions are interspersed in a stretch of contiguous bases.

14. The set of oligonucleotides of claim 13, wherein the pre-determined number of sample barcode positions varies among different sample barcodes.

15. The set of oligonucleotides of claim 13, wherein the barcode comprises about 10 to about 35 nucleotides.

16. The set of oligonucleotides of claim 13, wherein the barcode comprises about 12 to about 25 nucleotides.

17. The set of oligonucleotides of claim 13, wherein the sample barcode comprises 2, 3, 4, 5, 6, 7, 8, 9, 10, 11, 12, 13, 14, 15, 16, 17, 18, 19, or 20 sample index positions, or a combination thereof.

18. The set of oligonucleotides of claim 13, wherein the sample barcode comprises about 4 to about 12 sample index positions.

19. The set of oligonucleotides of claim 13, wherein the molecular barcode comprises about 5 to about 25 molecular index positions.

20. The set of oligonucleotides of claim 13, wherein the molecular barcode comprises about 5 to about 15 molecular index positions.

21. The set of oligonucleotides of claim 13, wherein sample index position nucleotides and molecular index position nucleotides are selected from:
(A) the sample index position nucleotide is A and the molecular index position nucleotide is C, G, T, or a combination thereof;
(B) the sample index position nucleotide is T and the molecular index position nucleotide is C, G, A, or a combination thereof;
(C) the sample index position nucleotide is C and the molecular index position nucleotide is G, A, T, or a combination thereof;
(D) the sample index position nucleotide is G and the molecular index position nucleotide is C, A, T, or a combination thereof;
(E) the sample index position nucleotide is A, T, or a combination thereof and the molecular index position nucleotide is C, G, or a combination thereof;
(F) the sample index position nucleotide is A, C, or a combination thereof and the molecular index position nucleotide is T, G, or a combination thereof;
(G) the sample index position nucleotide is A, G, or a combination thereof and the molecular index position nucleotide is T, C, or a combination thereof;
(H) the sample index position nucleotide is T, C, or a combination thereof and the molecular index position nucleotide is A, G, or a combination thereof;
(I) the sample index position nucleotide is T, G, or a combination thereof and the molecular index position nucleotide is A, C, or a combination thereof; or
(J) the sample index position nucleotide is G, C, or a combination thereof and the molecular index position nucleotide is A, T, or a combination thereof.

22. The set of oligonucleotides of claim 13, wherein each barcode comprises one or more additional index barcodes comprising index positions.

23. The set of oligonucleotides of claim 22, wherein the one or more additional index barcode is a cellular barcode, a barcode that provides a measure of DNA length of an unrepaired end, or both a cellular barcode and a barcode that provides a measure of DNA length of an unrepaired end.

24. The set of oligonucleotides of claim 13, wherein each oligonucleotide in the set of oligonucleotides further comprises non-barcode positions comprising sites for hybridization, sites for sequence primer binding, sites for amplification, or any combination thereof.

25.-71. (canceled)
